(19) United States
(12) Patent Application Publication
(10) Pub. No.: US 2024/0075184 A1
(43) Pub. Date: Mar. 7, 2024

(54) BIODEGRADABLE IMPLANTS WITH ANTICORROSIVE COATINGS

(71) Applicant: University of Georgia Research Foundation, Inc., Athens, GA (US)

(72) Inventors: Ramaraja P. Ramasamy, Athens, GA (US); Hamid Asadi, Athens, GA (US)

(21) Appl. No.: 18/272,765

(22) PCT Filed: Jan. 10, 2022
(86) PCT No.: PCT/US2022/070120

§ 371 Date: Jul. 17, 2023

Related U.S. Application Data
(60) Provisional application No. 63/139,890, filed on Jan. 21, 2021.

(51) Int. Cl. A61L 27/34; A61F 2/28; A61L 27/04; A61L 27/50; A61L 31/02; A61L 31/10; A61L 31/14

(52) U.S. Cl. CPC A61L 27/34; A61F 2/28; A61L 27/047; A61L 27/50; A61L 31/022; A61L 31/10; A61L 31/14; A61F 2002/30062

(57) ABSTRACT

Provided are biodegradable orthopedic implants, corrosion-resistant coating for orthopedic implants, and methods of making. The biodegradable orthopedic implant can include a Mg alloy having a coating including a first layer, a second layer, and a third layer. The first layer can be an oxide layer coated on an external surface of a magnesium alloy implant. The second layer can include lawsone embedded in PCL, and the third layer can include PCL. To manufacture the coated orthopedic implant, the implant can be immersed in an alkaline solution to form an oxide-coated implant and implant can then be coated with a PCL lawsone solution. An additional polymer protective layer can be added.

### CROSS-REFERENCE TO RELATED APPLICATIONS

This application claims priority to U.S. provisional application entitled “BIODEGRADABLE IMPLANTS WITH ANTICORROSIVE COATINGS,” having Ser. No. 63/139,890 filed on Jan. 21, 2021, which is entirely incorporated herein by reference.

### BACKGROUND

Temporary surgical implants, especially those made from magnesium alloys, are known for their biodegradation and biocompatibility, allowing for an implant that does not require secondary surgery for removal. However, magnesium alloy implants corrode rapidly under physiological conditions, often before bone healing can occur, leading to implant failure.

### SUMMARY

Embodiments of the present disclosure provide biodegradable orthopedic implants, corrosion-resistant coating for orthopedic implants, methods of making coated orthopedic implants, and the like.

An embodiment of the present disclosure includes a biodegradable orthopedic implant. The implant can include a Mg alloy and have a coating including PCL and lawsone.

An embodiment of the present disclosure also includes a corrosion-resistant coating for orthopedic implants, the coating including a first layer, a second layer, and a third layer. The first layer can be an oxide layer coated on an external surface of a magnesium alloy implant. The second layer can include lawsone embedded in PCL, and the third layer can include a polymer.

An embodiment of the present disclosure also includes method of making a coated orthopedic implant. The method can include immersing a magnesium alloy implant in an alkaline solution to form an oxide-coated implant and coating the oxide-coated implant with a PCL-lawsone solution to form a coated orthopedic implant.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1A is a diagram illustrating a three-layer coating in accordance with embodiments of the present disclosure. FIG. 1B is a diagram illustrating the behavior of the three-layer coating in accordance with embodiments of the present disclosure.

FIGS. 2A-2C show characteristics of bare, alkaline treated, and coated Mg samples in accordance with embodiments of the present disclosure. FIG. 2A provides SEM images, FIG. 2B provides ATR-FTIR spectra, and FIG. 2C provides water contact angle values.

FIG. 3A shows open circuit potential curves.

FIG. 3B shows potentiodynamic polarization curves of bare, alkaline treated, and coated Mg samples performed in Hank's solution.

FIGS. 4A-4D show EIS spectra of Mg samples performed in Hank's solution; Nyquist plots (FIG. 4A) and (FIG. 4B) (Insets: EC models employed for EIS data fitting), Bode-impedance plots (FIG. 4C), and Bode-phase plots (FIG. 4D).

FIGS. 5A-5D provide evolution of Bode plots spectra of the coated Mg samples performed in Hank's solution; Bode-phase plots of PCL (FIG. 5A) and Bode-impedance plots of PCL (FIG. 5B) (Insets: EC models employed for data fitting), Bode-phase plots of PCL-LS (FIG. 5C), and Bode-impedance plots of PCL-LS (FIG. 5D).

FIG. 6 provides SEM images of samples before and after 7 days of immersion in Hank's solution at 37° C. for 7 days. in accordance with embodiments of the present disclosure.

FIGS. 7A-7C show variation in hydrogen evolution volume (FIG. 7A), pH value (FIG. 7B) of each group during immersion in Hank's solution at 37° C. for 7 days and ATR-FTIR spectra (FIG. 7C) of samples after 7 days of immersion in Hank's solution.

FIG. 8 provides images of a Zone of Inhibition study.

FIG. 9 is a chart of results from a cytocompatibility study of the coating in accordance with embodiments of the present disclosure.

FIGS. 10A-10B are camera images of the PCL-LS coating in accordance with embodiments of the present disclosure on (FIG. 10A) untreated and (FIG. 10B) alkaline-treated AZ31 substrate before and after the cross-cut adhesion test.

FIGS. 11A-11D provide cross-sectional morphology of PCL-LS/substrate interface with the corresponding elemental analysis mapping and EDS spectra in accordance with embodiments of the present disclosure.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, material science, and the like, which are within the skill of the art.

The following examples are put forth so as to provide those of ordinary skill in the art with a complete disclosure and description of how to perform the methods and use the materials disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

Lawsone, as used herein, refers to (2-hydroxy-1,4-napthoquinone), which is a natural red-orange dye extracted from the leaves of Lawsonia inermis plant, commonly known as “henna”.

Polycaprolactone (PCL), as used herein, refers to a synthetic polymer that is a biodegradable polyester with a low melting point of around 60° C. and a glass transition temperature of about −60° C. Polycaprolactone is alternatively known as poly ε-caprolactone.

### General Discussion

In accordance with the purpose(s) of the present disclosure, as embodied and broadly described herein, embodiments of the present disclosure, in some aspects, relate to Magnesium alloy orthopedic implants including anticorrosive coatings.

In general, embodiments of the present disclosure provide for methods of making the anticorrosive coatings, coating compositions including PCL and lawsone, and biodegradable implants including the coating compositions.

The present disclosure includes biodegradable orthopedic implants having a coating that can include polycaprolactone (PCL) and lawsone. The implant can contain a magnesium alloy and can biodegrade over time as the surrounding bone heals. Advantageously, the coating can impart anti-corrosive properties to the implant, allowing the implant to biodegrade more slowly. The coating can have antibacterial properties and is noncytotoxic.

In some embodiments, the coating provided herein can have multiple layers. In some embodiments, the coating can have a first layer, a second layer, and a third layer. The first layer can be an oxide layer on an external surface of the implant. The second layer can be an inhibition layer including a corrosion inhibitor and a polymer. In an embodiment, the inhibition layer can include lawsone embedded in PCL. The third layer can be a polymer protective layer. In some embodiments the polymer protective layer can be or can include PCL. In some embodiments, the PCL can be substituted with or used in conjunction with other natural or synthetic polymers with suitable biocompatibility.

The first layer can be a dense oxide layer. After alkaline treatment, the dense oxide layer is formed on the surface of a Mg alloy to protect the highly susceptible Mg alloy surface against corrosion during the coating procedure. In some embodiments, the oxide layer can be about 4 μm. The dense oxide layer can be formed by immersion of the implant in an alkaline solution. In some embodiments, the alkaline solution is NaOH. The NaOH can have a concentration of about 0.1-5 M, and the immersion time can be about 1-24 hours, depending on the concentration.

A second layer including PCL and lawsone forms the middle layer. Lawsone is initially entrapped within the second layer of the coating as the corrosion inhibitor. A third, top PCL layer can be coated on the second as a barrier layer to minimize the leaching of the inhibitor. Advantageously, once a defect appears within the PCL coating, the lawsone molecule is released from the inner layer into the damaged area. The anticorrosive properties of lawsone are due to its ability to chelate Mg²⁺ metal cations from the Mg alloy to form a protective barrier. While the functional PCL-lawsone layer is adjacent to the Mg substrate, in some embodiments additional layers of PCL can be added on top of the functional layer to increase the passive barrier properties of the coating.

In some embodiments, the PCL and PCL-lawsone layers can be about 5 μm each. In some embodiments, the layer or layers comprising PCL and lawsone can include about 0.1% w/w to 3% w/w, about 0.5% w/w to 1.5% w/w, or about 1% w/w of lawsone to PCL. In a particular embodiment, a ratio of about 1% w/w lawsone to PCL can provide both antibacterial and anticorrosion properties. However, a lower concentration of lawsone can also be used as a corrosion inhibitor but would not show notable antibacterial properties.

In some embodiments, the corrosion inhibitor (e.g. lawsone) is directly added into the coating (e.g. PCL). In other embodiments, the lawsone and/or other corrosion inhibitors can be encapsulated in micro/nanoparticles and then incorporated into the polymeric coating to minimize the undesired leaching or obtain stimuli-responsive release of the inhibitor. Lawsone can be encapsulated in various micro/nanoparticles including organic (polymeric particles such as carboxymethylcellulose, chitosan, PCL, polyurethane, polyelectrolyte nanocapsules, polystyrene containers, etc.) and inorganic (such as halloysites, mesoporous silica, mesoporous TiO₂, layered double hydroxides, hydroxyapatite, etc.) nanocontainers. The encapsulation can provide protection of the inhibitor by preventing uncontrollable release or undesired chemical reactions with the coating material and has been shown to increase the corrosion protection of the substrate when compared to directly adding the inhibitor into the coating material. The inhibitor chemical agent remains sequestered and protected within the capsule until released by damage to the coating.

The implant can be biodegradable implant, such as a Mg—Al—Zn alloy. In some embodiments, the Mg—Al—Zn alloy can have an Al content of about 3% to 13%, about 2.75% to 5%, or about 3%. In a particular embodiment, the Mg—Al—Zn alloy can be AZ31.

Described herein is an anti-corrosive polymeric coating as above, which includes a synthetic polymer and lawsone. The coating can be included on temporary orthopedic implants, such as implants comprising magnesium alloys.

Embodiments of the present disclosure also provide for methods of making coated orthopedic implants as above. The method can include immersing a magnesium alloy implant in an alkaline solution to form an oxide-coated implant and coating the oxide-coated implant with a PCL-lawsone solution. A layer including PCL can be coated on the top.

Advantageously, the coating described herein can prevent, delay and/or mitigate formation of hydrogen-forming gas pockets around the implant.

The implants described herein can be such as a screw, a plate, a nail, a pin, a rod, or a prosthesis, as can be envisioned by one of ordinary skill in the art.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

Magnesium (Mg) alloys have emerged as a new class of biodegradable metallic biomaterials in recent years. Especially, they have gained extensive attention as potential temporary orthopedic bone implants such as fixation screws, plates and pins for the healing of bone defects owing to their biocompatibility and biodegradability, and mechanical properties suitable to natural human bones. In the physiological environment, Mg alloy can be degraded and safely adsorbed by the human body with no inflammatory responses, while the damaged bone tissue is being reconstructed and substituted with them. This eliminates the need for a second surgery to remove the implant from the body after full healing of the bone defect.

Despite all the desirable properties of Mg alloys, their practical application is still limited because of their poor corrosion resistance and rapid degradation rate [5]. Fast corrosion results in excess H₂ generation at the implant site, local alkalization, and deteriorated mechanical integrity of the implant which may delay the healing process and even cause tissue necrosis [6-8]. Together, these issues emphasize the need for novel strategies to tailor the corrosion/degradation rate of Mg alloys and expand their clinical application as bioimplants.

Numerous corrosion protection methods including surface chemical treatment [9], phosphate coating [10], anodization [11], micro-arc oxidation [12], and polymeric coating [13] have been adopted for Mg alloys so far. Among them, polymeric coatings have been recognized as the most effective method to tailor the corrosion rate of Mg alloys without changing their bulk properties [14]. Besides, they can provide other functionalities to the substrate such as enhanced biocompatibility and cellular responses, ability to load small molecules, self-healing, and antibacterial properties [13].

Several synthetic polymers such as polylactic acid (PLA) [15], poly(lactic-co-glycolic) acid (PLGA) [16], polydopamine [17], and polycaprolactone (PCL) [18] as well as natural polymers like chitosan [19] and silk fibroin [7] have been used as protective coatings on Mg alloys in biomedical applications. Although these polymeric coatings can improve the corrosion resistance of Mg alloys to a certain degree, they are susceptible to damage by the highly corrosive environment of the human body, which results in polymer degradation and deterioration of protective barrier properties [20]. Following the emergence of defects or micro-cracks within a coating, corrosive species and water penetrate through the defected areas of the coating and reach the Mg alloy surface, leading to the occurrence of localized corrosion (mainly pitting corrosion) and rapid degradation of Mg alloys [21].

Introducing corrosion inhibitors into the polymeric coatings have shown great potential to improve the anti-corrosion properties and durability of the polymeric coatings [22]. Generally, a composite polymer/corrosion inhibitor coating consists of a polymeric coating as a passive physical barrier, hosting an active corrosion inhibitor within the coating matrix. When the barrier coating is damaged by corrosive species, the loaded corrosion inhibitor can be released into the defected sites and mitigate the corrosion progression by forming insoluble metal complexes [23, 24]. Several inorganic and organic corrosion inhibitors such as cerium ion [25], 8-hydroxyquinoline [26], vanadate [27], molybdate [28], and benzotriazole [29] have been proposed for corrosion protection of Mg alloys so far. Although being effective in corrosion mitigation, most of these synthetic inhibitors are not suitable for implantable biomedical applications due to their potential cytotoxic and carcinogenic effects in the physiological environment [30, 31].

Eco-friendly corrosion inhibitors also known as “green” inhibitors have emerged as promising alternatives to hazardous synthetic inhibitors [32]. Among all the green alternatives, plant-derived corrosion inhibitors have gained considerable attraction, as they possess desired biocompatibility while being inexpensive and available through renewable resources [33, 34]. Lawsone (2-hydroxy-1,4-napthoquinone) is a natural red-orange dye, extracted from the leaves of Lawsonia inermis plant, commonly known as “Henna”. As the main active ingredient of Henna, lawsone has well-known corrosion inhibition properties based on the chelation of metal cations [35, 36]. It has been used as a corrosion inhibitor for different metal substrates such as steel and aluminum alloys in non-medical applications, by either adding it to a corrosion solution or incorporating it in a coating formulation [37, 38]. In a comprehensive study by Lamaka et al. [39], inhibition efficiencies of multiple corrosion inhibitors on Mg and its alloys were examined through hydrogen evolution test. Lawsone (0.4 M in corrosion solution) showed excellent inhibition efficiencies of 97% and 90% for high-purity Mg (Fe % 51 ppm) and Mg alloy AZ31, respectively.

In addition to the corrosion inhibition properties, lawsone is non-toxic to the human body and has been widely used in various biomedical applications such as wound healing [40], antibacterial coating [41], and cancer treatment [42]. Moreover, it has been reported that lawsone possesses antibacterial and antibiofilm activities against gram-positive and gram-negative bacteria [43]. Such antibacterial effects can be helpful in the prevention of microbial infection and failure of the implant especially at early stages after implantation [44]. Taken together, lawsone is an attractive candidate to be used in the fabrication of protective coatings on biodegradable Mg-based orthopedic implants. To date, lawsone has not been considered for use as an anticorrosive inside the human body.

### Example 1

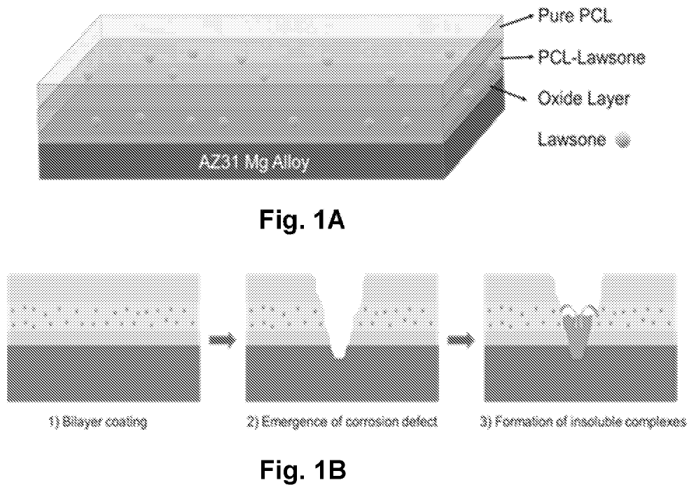

Described herein is a composite coating on a Mg alloy for biodegradable bone implant application. The studies provided herein demonstrate the first time lawsone has been incorporated into a coating formulation to improve the corrosion resistance properties of an Mg-based alloy. The structure of the proposed composite coating on the AZ31 Mg substrate is schematically presented in FIGS. 1A and 1B. AZ31 is one of the Mg—Al—Zn alloys. Having a comparatively lower Al content (˜3 wt %) than other magnesium alloys (with typical Al contents of 3-13 wt %), makes AZ31 more suitable for biomedical applications, as excessive Al contents may be harmful to neurons and the osteoblast cells [45, 46]. Before coating, the AZ31 substrate was alkaline treated to form a temporary passive layer of Mg(OH)₂ on the surface. This layer protects the active surface of AZ31 against corrosion during the coating process and ensures the formation of a homogenous coating with no bubbles and pores [47]. PCL, a bioresorbable semi-crystalline polyester, was selected as the polymeric matrix in the proposed coating, due to its excellent biocompatibility and slow degradation rate [48]. Previous studies have also shown that PCL coatings can effectively hinder the initial rapid degradation of Mg alloys [49]. The disclosed coating has a bi-layered structure consisting of a PCL/lawsone inner layer and a pure PCL top layer. The top layer serves as a physical barrier to minimize the excessive leaching of the loaded inhibitor. The PCL/lawsone is the functional layer of the coating, in which lawsone was embedded as a natural and non-toxic corrosion inhibitor, to improve the corrosion inhibition ability. Such bilayer coating structure benefits form the biocompatibility and barrier properties of PCL, and the corrosion inhibition and antibacterial activity of lawsone. The morphological and physicochemical properties of the fabricated coating were characterized by SEM, ATIR-FTIR, and water contact angle measurement. The corrosion protection effect of the coating was assessed by electrochemical tests and immersion experiments in Hank's solution. Finally, the antibacterial properties and cell compatibility of the coating were evaluated.

### Materials and Methods

Chemicals and Reagents—Poly(ε-caprolactone) (PCL) with the average molecular weight of 80,000 g/mol and lawsone (2-Hydroxy-1,4-naphthoquinone, 97%) were purchased from Sigma-Aldrich. Dichloromethane was obtained from EMD chemicals. All other reagents, unless otherwise specified, are of reagent grade. Milli-Q water (resistivity=18 MΩ.cm) was used for the preparation of all electrolytes and aqueous solutions.

Preparation of AZ31 Substrates—AZ31 Mg alloy sheet with a thickness of 1 mm was cut into the pieces of 2×2 cm. AZ31 samples were first mechanically polished by 2000-grit silicon carbide abrasive paper to remove contamination and native oxides layer. Then, they were ultrasonically cleaned in ethanol and distilled water each for 5 min, and finally dried in an oven at 60° C. The chemical composition of the AZ31 alloy is shown in Table 1.

TABLE 1
Elemental composition of AZ31 Mg alloy (mass fraction, %).

| Al | Zn | Mn | Si | Cu | Fe | Ni | Others | Mg |
|---|---|---|---|---|---|---|---|---|
| 2.5-3.5 | 0.7-1.3 | 0.2-1 | 0.5 | 0.01 | >0.5 | >0.5 | 0.4 | remaining |

Alkaline Pretreatment—Before applying the polymeric coating on the Mg alloy surface, it is necessary to provide the Mg sample with primary protection against possible corrosion during the coating process. In this study, alkaline treatment was used to form a passive layer of Mg(OH)₂ on the Mg surface to enhance its corrosion resistance. To do so, all Mg samples were immersed in 1 M NaOH solution at 80° C. for 4 h. After the reaction was completed, samples were thoroughly rinsed with deionized (DI) water and dried at 80° C. This sample was labeled as “AZ31-OH”.

Fabrication of PCL-Lawsone Coatings—PCL solution (1% w/v) was prepared by dissolving PCL in dichloromethane using a magnetic stirrer for 1 h. Lawsone powder was added to the PCL solution and stirred to obtain a homogenous solution with 1% w/w of lawsone with respect to PCL weight. The resulting PCL/lawsone solution (200 μL) was pipetted on alkaline pretreated Mg samples and dried under the hood for 24 h at room temperature. During the drying process, all samples were placed on glass Petri dishes with closed lids to control the solvent evaporation rate. The same process was repeated for the other side of the samples. To prepare the top coating, a pure PCL solution (200 μL) was pipetted on both sample sides. The lawsone-interbedded samples were labeled as “PCL-LS”. Meanwhile, specimens coated with pure PCL were also prepared as controls under the same condition and denoted as “PCL”.

Coating Characterization—The surface and cross-section morphologies of the coatings were visualized by a scanning electron microscope (SEM, FEI Teneo, FEI Co.). Before SEM observation, the samples were gold-sputtered to improve conductivity. The functional groups of the coatings were recognized by attenuated total reflectance-Fourier transform Infrared analysis (ATR-FTIR, Nicolet 6700, Thermo Electron Corporation, MA, USA). The spectra were recorded from 4000 to 800 cm⁻¹ in wavenumber and 128 scans with a resolution of 4 cm⁻¹. The surface hydrophilicity of samples was measured using a Krüss DSA 100 drop shape analyzer at room temperature. The static contact angle was measured by the dropwise addition of distilled water (1 μL) onto the sample surface.

In vitro Corrosion Evaluation: Electrochemical Measurements—Hank's solution (compositions are listed in Table 2) was used for electrochemical and immersion experiments (according to ASTM-G31-72) to mimic the main inorganic components of the

corrosive aqueous media in the human body [50]. Electrochemical measurements were conducted in a custom-made corrosion cell in Hank's solution using CHI-920c model potentiostat (CH Instruments Inc., Austin, TX). The set-up consists of three electrodes with Mg samples exposing a surface area of 1 cm² as the working electrode, a silver/silver chloride (Ag/AgCl 3 M) reference electrode, and a platinum wire as the counter electrode. First, the open circuit potential (OCP) values of samples were recorded for 30 min without any external disturbance. Then, electrochemical impedance spectroscopy (EIS) measurement was carried out at OCP between frequencies of 10⁻² to 105 Hz with an AC amplitude of ±10 mV. Immediately, after the EIS test, a potentiodynamic polarization (PP) test was performed from −250 mV to +250 mV vs OCP at a scan rate of 1 mV/s to evaluate the corrosion behavior of the coatings. The EIS data were quantitatively simulated using appropriate equivalent circuit (EC) models. EIS measurements of coated samples after 1, 3, and 7 days of immersion in Hank's solution were also carried out under the same conditions. All electrochemical corrosion experiments were carried out in triplicates, and the average values was used for analyses. The corrosion potential (Ecorr) and corrosion current density (Icorr) were determined from PP curves by Tafel extrapolation method. The inhibition efficiency (IE) of each sample was calculated using Eq. 1 as below [51]:

$$\% IE = \frac{{I}_{o} - {I}_{i}}{{I}_{o}} \times 100 \qquad ( Eq . 1 )$$

Where Iₒ and Iᵢ are the corrosion current densities of AZ31 (control sample) and the tested sample, respectively.

TABLE 2
Composition of Hank's solution used for in vitro corrosion studies.

| | | | | | | | D- | |
|---|---|---|---|---|---|---|---|---|
| NaCl | KCl | CaCl₂ | MgSO₄•7H₂O | MgCl₂•6H₂O | Na₂HPO₄•2H₂O | KH₂PO₄ | Glucose | NaHCO₃ |
| 8 g/L | 0.4 g/L | 0.14 g/L | 0.1 g/L | 0.1 g/L | 0.6 g/L | 0.6 g/L | 1 g/L | 0.35 g/L |

Immersion Experiments—Samples were immersed in a sealed vial containing Hank's solution at 37° C. with a volume-to-sample area ratio of 20 mL/cm² (ASTM G31-72). For each sample, the volume of evolved hydrogen generated by Mg samples was measured every 24 h up to 7 days. The experimental set-up for this test consists of an inverted funnel-burette system above the sample, immersed in a solution-filled container. Meanwhile, the pH values of the immersion solutions were recorded during the immersion test. After immersion for 7 days, all samples were taken out and thoroughly rinsed with DI water to remove the corrosion products on the surface and quickly dried. Post-corrosion surface morphology was characterized using SEM and surface compositions were analyzed using ATR-FTIR analysis.

Antibacterial Activity Assay—Escherichia coli (E. coli) and Staphylococcus aureus (S. aureus) bacterial strains were used to study the antibacterial activities of the coatings using the Kirby-Bauer disk diffusion test [52]. Briefly, a pure bacterial culture is suspended in a phosphate buffer saline (pH 7.4), adjusted to a turbidity of 0.5 McFarland standard (equal to the density of a bacterial suspension with a 1.5×10⁸ colony forming units (CFU/ml)), and swabbed uniformly across a culture plate. Mg samples were cut into pieces of 1×1 cm in diameter, sterilized under UV light for 1 h each side, and placed on the culture plates. After, incubating the plates at 37° C. for 24 h, the antibacterial activity was evaluated by measuring the diameter of the inhibitory zone formed around the samples.

Cytocompatibility: Cell culture—Fetal Osteoblast cells (hFOB 1.19-ATCC 11372) were cultured in 75 cm² T-flasks with a 1:1 mixture of Ham's F12 Medium and Dulbecco's Modified Eagle's Medium, containing 0.3 mg/mL Geneticin (Gibco Life Technologies) and supplemented by 10% fetal bovine serum (FBS) in a humidified incubator at 37° C. with 5% CO₂. The culture medium was replenished with fresh media every 2 days until cells were 90% confluent. Thereafter, the cells were detached from the T-flask surface by enzymatically degrading their extracellular matrix layer by treating them with 0.18% trypsin and 5 mM EDTA for 5 min.

Cell Viability—The effect of leach outs from the Mg samples in hFOB media were tested against Fetal Osteoblast cell proliferation. The sample leachates were obtained using cell culture media with an extraction media/surface area ratio of 1.25 mL/cm² in a humidified atmosphere with 5% CO₂ at 37° C. for 1,3, and 5 days according to ISO 10993-12 [53]. Around 5000 cells/mL were seeded in a cell culture grade 96-well plate and incubated for 24 h in a humidified incubator with 5% CO₂ at 37° C. The manufacturer's protocol (Sigma-Aldrich) was followed to perform the cytocompatibility test using a CCK-8 kit on hFOB. After 24 h of cell culture incubation in a 96-well plate, 10 μL of the leachates (1.25 mL/cm2) from the samples were added (n=6) to the cells. The cells were allowed to respond to the leachates during a separate 24 h incubation period inside a cell culture incubator at physiological temperature. Then, 10 μL of the WST-8 solution was added to the resulting solution and incubated for 2 h. During this time, dehydrogenase enzymes from live cells acted on the WST-8 solution, converting it to an orange product, formazan, measurable at 450 nm. The relative viability (%) of the cells in response to sample leachates was reported relative to the control (without leachate exposure) using Eq. 2:

$$\% cell viability = \frac{absorbance of the test samples}{absorbance of the control samples} \times 100 \qquad ( Eq . 2 )$$

Statistical analysis—All data in this study were expressed as mean±standard deviation with n=3. One-way analysis of variance (ANOVA) tests was used to determine statistical significance, where significance was defined as p<0.5.

### Results

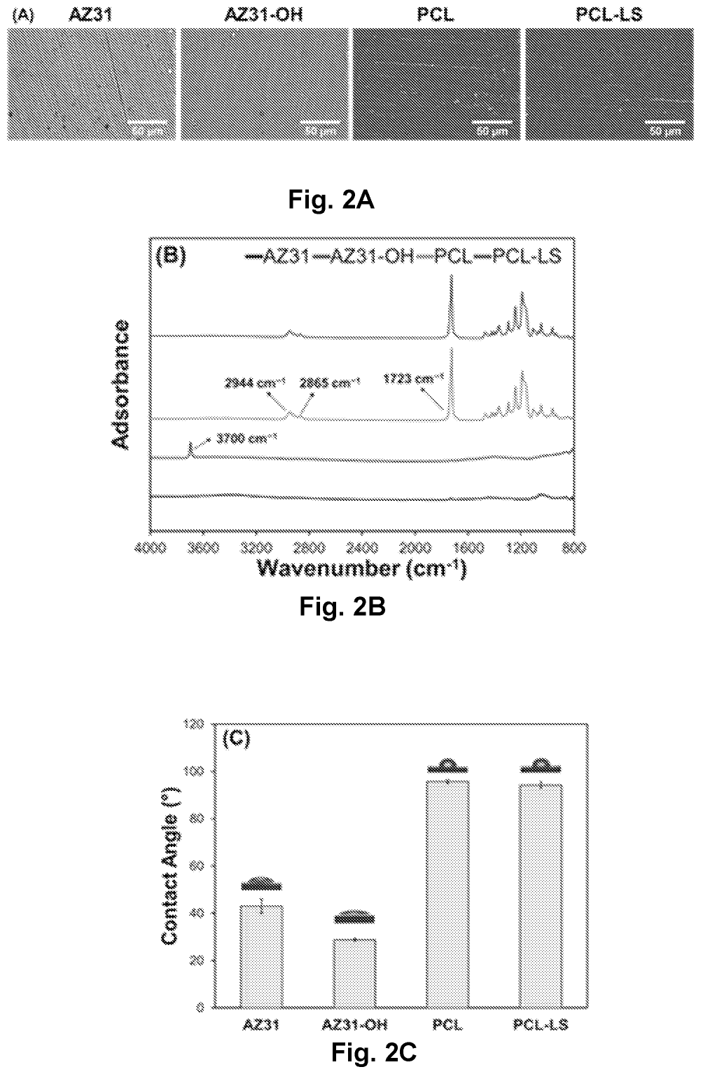

Coating Characterizations: Surface Morphology—FIG. 2A exhibits the SEM surface morphologies of as-prepared Mg samples. The bare AZ31 substrate showed a flat and smooth surface with some orderly aligned scratches on that, remained from the mechanical polishing step. After alkaline treatment and formation of a dense oxide layer, the scratches became shallower on AZ31-OH surface. The coated Mg samples (both PCL and PCL-LS) evidently exhibited smooth and uniform polymeric layers on their surfaces, which was distinct from the surface morphologies of those uncoated Mg samples. Moreover, no obvious defects were observed on the coating, suggesting that the coatings can fully cover the substrates and protect them against corrosive environment.

Chemical Composition of the Coatings—ATR-FTIR was employed to analyze the chemical compositions of the sample surfaces resulted from pretreatment and coatings. As shown in FIG. 2B, the bare AZ31 surface did not show any distinctive peak, indicating that no chemical groups were present on the surface. After alkaline treatment, a sharp peak at 3700 cm⁻¹ corresponding to hydroxyl groups appeared in the spectrum, showing that the Mg(OH)₂ layer was formed on the Mg alloy surface [54]. Formation of the oxide layer is necessary to protect the highly susceptible Mg alloy surface against corrosion during the coating procedure. Moreover, the emerged hydroxyl groups on the Mg alloy surface may enhance the adhesion strength of the coatings to the substrate through chemical interaction with coating molecules [47]. The FTIR spectrum of PCL coating presented the characteristic absorption peaks of PCL. The peaks at 2944 cm⁻¹, 2865 cm⁻¹, and 1723 cm⁻¹ were assigned to asymmetric CH₂ stretching, symmetric CH₂ stretching, and C═O stretching of PCL, respectively, verifying the presence of PCL coating [55]. The spectrum of PCL-LS composite coating did not show discernible differences compared to the pure PCL spectrum. This might be due to the presence of pure PCL top coatings and a limited penetration depth of ATR-FTIR (less than 1 μm), suppressing the emergence of lawsone characteristic peaks in the spectrum [56].

Water Contact Angle—The hydrophilicity of the Mg samples was investigated by the water contact angle measurement test. FIG. 2C shows the measured water contact angles and their representative images. Pristine AZ31 Mg alloy showed a relatively hydrophilic surface with a contact angle of ˜43°. After alkaline treatment, the water contact angle was reduced to ˜29° due to the presence of abundant hydroxyl groups of the formed Mg(OH)₂ layer on the AZ31 surface, showing improved hydrophilicity [57]. In contrast, PCL coated Mg samples clearly exhibited a less hydrophilic surface (nearly round-shaped droplet) with a contact angle value of ˜96° compared to the uncoated Mg samples. The lawsone entrapment in the PCL (PCL-LS) brought about no significant difference in contact angle, since the PCL top coating is the one that interacts with water molecules. The results of surface morphologies, ATR-FTIR, and contact angle measurement confirmed that all surface modification and coating steps were successfully carried out on the Mg alloy surface.

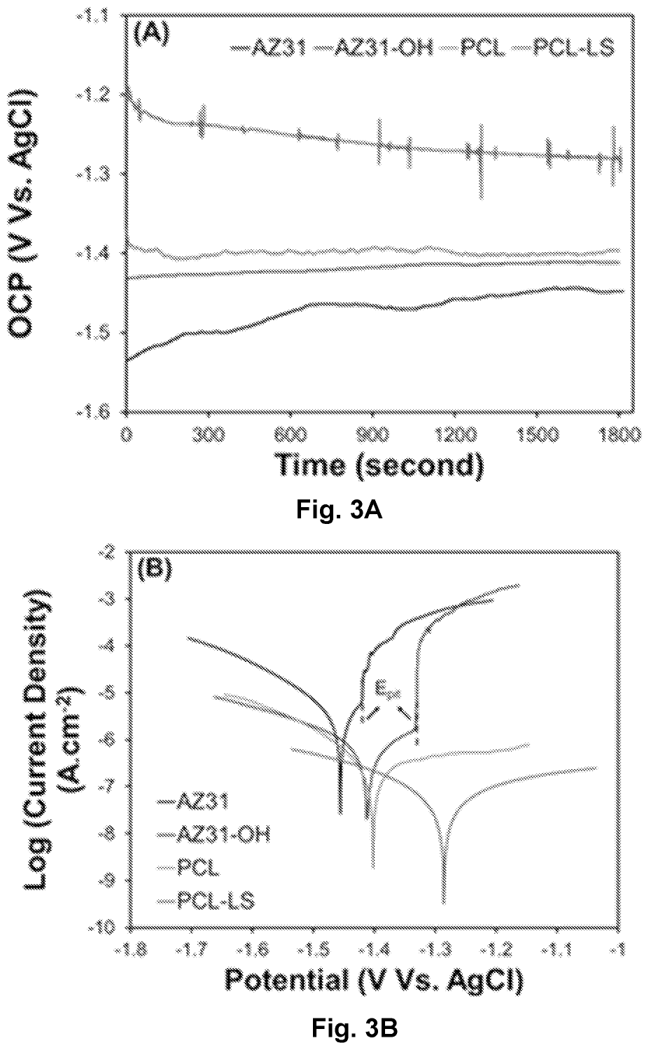

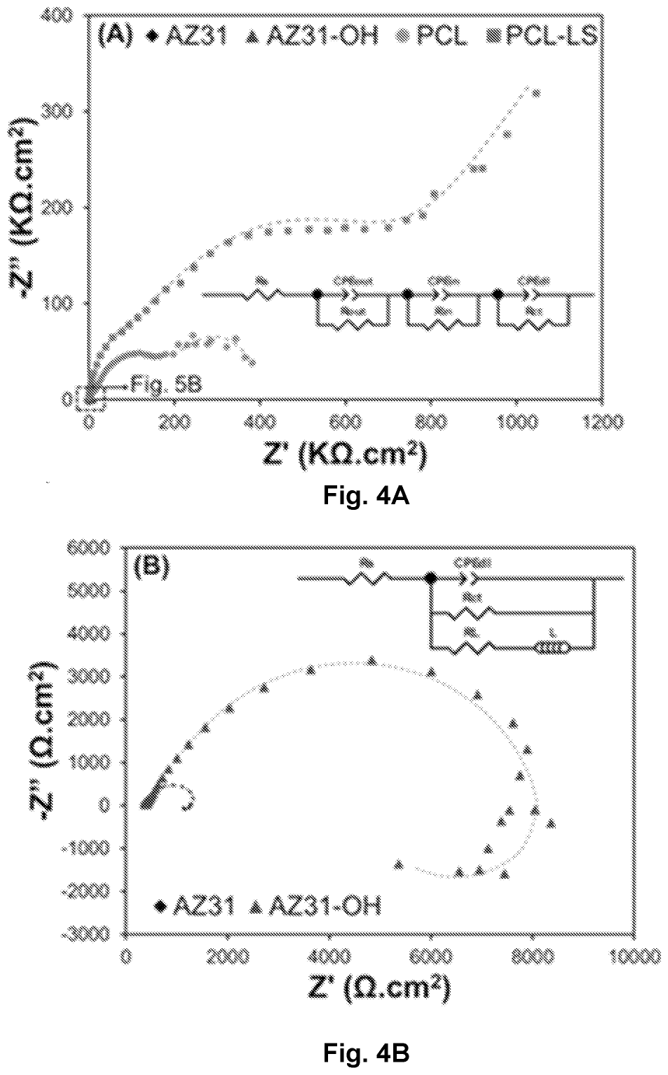

In vitro Corrosion Studies: Open Circuit Potential and Polarization Measurements—The electrochemical measurements were performed in Hank's solution to further investigate the corrosion resistance of each sample using OCP and PP (Tafel plot) measurements. FIG. 3A displays the OCP spectra of all samples for 30 min. The OCP curves reflect the potential change of the electrode surface in electrolyte without externally applied voltage [58]. Generally, a more positive OCP value means the surface is nobler and therefore, less susceptible to corrosion [59]. Bare AZ31 sample showed some fluctuation and finally stabilized at a quite cathodic/negative OCP value of −1.448 V. The fluctuating of OCP is associated with the active corrosion and product formation/dissolution taking place on the AZ31 surface [6]. The final OCP of AZ31-OH and the PCL increased to more positive values of −1.412 and −1.401, respectively, implying the more stable surfaces of these samples compared to the AZ31 sample. The OCP values were further going up toward more positive value by adding the inhibitor to the PCL coatings and reached to the highest value of −1.279 V for the PCL-LS coating. The more positive OCP values achieved by the incorporation of lawsone can slow down the cathodic hydrogen evolution and subsequently, the overall rate of corrosion reaction [60].

Potentiodymanic polarization curves can provide invaluable information about the corrosion process as well as the corrosion rate of the substrate being tested. The Tafel curves of AZ31, alkaline treated, and coated samples are shown in FIG. 3B, and the corresponding values of E_{corr}, I_{corr}, and IE are listed in Table 3. The pristine AZ31 substrate exhibited a typical curve of active metals corrosion with a low E_{corr} of −1.456 V and a high I_{corr} of 3.71×10⁻⁶ A.cm⁻². After alkaline treatment, the curve of AZ31-OH slightly shifted toward more positive potentials and showed a decreased I_{corr} of 8.92×10⁻⁷ A.cm⁻². This is mainly because of the formed Mg(OH)₂ protective layer that can isolate the bulk substrate from the corrosion medium. The presence of sharp current changes on the polarization curves of AZ31 (at −1.41 V) and AZ31-OH (at −1.331 V), indicated the breakdown of the substrates due to the possible pitting corrosion [47]. No sign of pitting corrosion was observed on the Tafel plots of coated samples. The PCL coated sample showed an I_{corr} value of 5.34×10⁻⁷ A.cm⁻², which was much lower than that of the uncoated Mg substrates. This is because of the physical barrier effect of the PCL coating against the penetration of the corrosive electrolyte, resulting in enhanced corrosion resistance of the sample [61]. Incorporation of lawsone into the PCL coating, remarkably shifted the E_{corr} values toward a more positive potential of −1.288 V. A more positive E_{corr} values implies that the surface has become nobler and therefore, higher potential driving forces are required to initiate the corrosion on the sample [62]. This suggests that the addition of the inhibitor can further improve the corrosion resistance of the PCL coating. As listed in Table 3, PCL-LS exhibited the highest IE of 98.3% and the lowest I_{corr} of 5.98×10⁻⁸ A.cm⁻² among all samples, which is about two orders of magnitude lower than that of AZ31.

TABLE 3
The corrosion potentials (E_{corr}), corrosion current densities
(i_{corr}), and corrosion inhibition efficiency (IE) of the Mg samples
obtained from PP curves in Hank's solution by Tafel methods.

| Samples | E_{corr} (V) | I_{corr} (A/cm²) | IE (%) |
|---|---|---|---|
| AZ31 | −1.45 | 3.7 × 10⁻⁶ | — |
| AZ31-OH | −1.41 | 8.9 × 10⁻⁷ | 75.9 |
| PCL | −1.39 | 5.3 × 10⁻⁷ | 85.6 |
| PCL-LS | −1.28 | 5.9 × 10⁻⁸ | 98.3 |

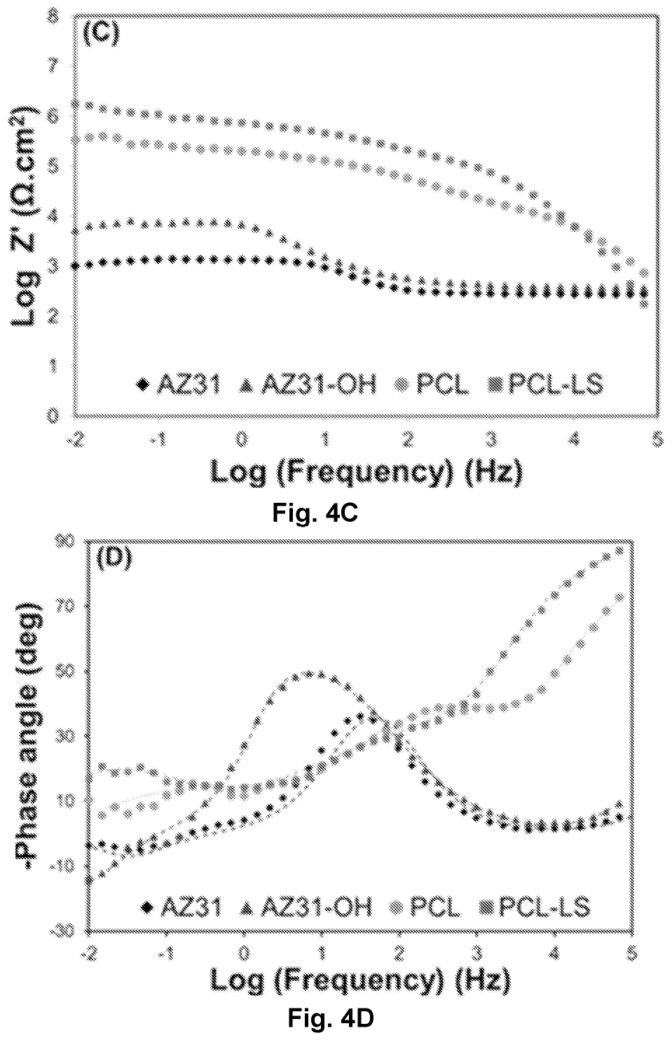

EIS measurements—EIS was utilized as a powerful technique to further investigate the corrosion protection performance of the coatings and corrosion mechanism [63]. Nyquist plots and Bode plots (impedance and phase angle plots) of EIS spectra for the tested samples are presented in FIG. 4. Generally, the diameter of the semicircle in the Nyquist plot has been associated with the corrosion resistance of a sample, wherein a larger arc means superior corrosion protective properties [64]. As shown in FIG. 4A-B, the PCL showed a much larger capacitive loop compared to that of AZ31 and AZ31-OH samples, indicating the enhancement of corrosion resistance by applying the PCL coating on AZ31 surfaces. The diameter of the capacitive loop of PCL-LS was furthered increased, suggesting that the incorporation of lawsone enhances the corrosion resistance of the coating more effectively than pure PCL. It is well known that the value of impedance modulus at the lowest-frequency region (|Z|_{0.01 Hz}) of the Bode impedance plot can be used as a semi-quantitative indicator of the anti-corrosive properties of a sample [65]. As shown in FIG. 4C, the |Z|_{0.1 Hz} values of the tested samples are in the following order: AZ31<AZ31-OH<PCL<PCL-LS, which was consistent with the I_{corr} values obtained from PP tests. From the Bode-phase plots in FIG. 4D, it can be noted that the broad peak at medium-frequency range is more pronounced for AZ31-OH that that of bare AZ31, which reflects the improvement of the oxide layer [66]. Besides, both PCL and PCL-LS showed high phase angles at the initial high-frequency region, which are generally attributed to the coating layers and indicate the barrier performance of the coatings[67]. Compared with PCL coating, PCL-LS exhibited a broader high-frequency phase and kept higher impedance values over a wider frequency range, which indicates the superior barrier performance of the PCL-LS coating [68]. Two equivalent electrical circuits (EECs) applied to fit the EIS data are shown in FIG. 4A-B (insets). In the EEC used for AZ31 and AZ31-OH, Rₛ and R_{ct} represent the electrolyte and interfacial charge transfer resistance, while CPE_{dl} reflects the capacitive behavior of the electric double layer. Besides, an L-R_{L} component was implemented to reflect the pseudo-inductive behavior at the low-frequency region associate with dissolution and pitting corrosion [69]. For the PCL and PCL-LS samples with thick coating layers, the resistance and capacitive behavior of the coatings were divided into outer and inner parts which were shown by Rₒᵤₜ/CPEₒᵤₜ and Rᵢₙ/CPEᵢₙ, respectively. In all EEC models, constant-phase elements were introduced instead of ideal capacitors to take the intrinsic inhomogeneity of the coating and metal surface into account and minimize the fitting error [70]. Each CPE element consists of two other parameters; Y0 (admittance) and n (exponent) [71]. The obtained electrochemical parameters from each model are presented in Table 4. As listed in Table 4, the PCL-LS showed the highest value of R_{ct} (50.2 kΩ.cm²) and the lowest value of CPE_{dl} (2.20×10⁻⁹ F.cm²) among all samples, suggesting the best corrosion protection performance of PCL-LS [26].

TABLE 4
Representative fitting results of EIS spectra of Mg samples using appropriate equivalent electrical circuits.

| | | Rₒᵤₜₑᵣ | Qₒᵤₜₑᵣ | | Rᵢₙₙₑᵣ | Qᵢₙₙₑᵣ | | R_{ct} | Q_{dl} | | R_{L} | L | |
|---|---|---|---|---|---|---|---|---|---|---|---|---|---|
| | Time | (Ω · | (F · | | (Ω · | (F · | | (Ω · | (F · | | (Ω · | (H · | |
| Samples | (day) | cm²) | cm⁻²) | n₁ | cm²) | cm⁻²) | n₂ | cm²) | cm⁻²) | n₃ | cm²) | cm⁻²) | Chi² |
| AZ31 | — | — | — | — | — | — | — | 1346 | 5.81 × | 0.78 | 1941 | 361 | 0.0067 |
| | | | | | | | | | 10⁻⁵ | | | | |
| AZ31- | — | — | — | — | — | — | — | 6822 | 9.80 × | 0.60 | 4232 | 12479 | 0.0206 |
| OH | | | | | | | | | 10⁻⁶ | | | | |
| PCL | — | 1.84 × | 2.99 × | 0.96 | 2.30 × | 7.59 × | 0.61 | 15612 | 3.98 × | 0.96 | — | — | 0.0047 |
| | | 10⁵ | 10⁻⁷ | | 10⁵ | 10⁻⁶ | | | 10⁻⁹ | | | | |
| | 1 | 94370 | 5.61 × | 0.52 | 23040 | 2.46 × | 0.64 | 13501 | 2.00 × | 0.96 | — | — | 0.0005 |
| | | | 10⁻⁶ | | | 10⁻⁷ | | | 10⁻⁹ | | | | |
| | 3 | 91572 | 4.45 × | 0.27 | 23033 | 7.69 × | 0.82 | 10928 | 4.56 × | 0.88 | — | — | 0.0013 |
| | | | 10⁻⁵ | | | 10⁻⁶ | | | 10⁻⁹ | | | | |
| | 7 | 52582 | 1.56 × | 0.28 | 13057 | 8.13 × | 0.97 | 5849 | 9.68 × | 0.68 | — | — | 0.0074 |
| | | | 10⁻⁸ | | | 10⁻⁶ | | | 10⁻⁶ | | | | |
| PCL- | — | 2.48 × | 2.01 × | 0.53 | 5.24 × | 6.52 × | 0.69 | 50236 | 2.20 × | 0.94 | — | — | 0.0044 |
| LS | | 10⁶ | 10⁻⁶ | | 10⁵ | 10⁻⁸ | | | 10⁻⁹ | | | | |
| | 1 | 6.84 × | 2.16 × | 0.47 | 3.96 × | 6.77 × | 0.68 | 33317 | 1.14 × | 0.99 | — | — | 0.0028 |
| | | 10⁶ | 10⁻⁶ | | 10⁵ | 10⁻⁸ | | | 10⁻⁹ | | | | |
| | 3 | 1.17 × | 2.89 × | 0.59 | 1.43 × | 1.47 × | 0.61 | 21126 | 8.61 × | 0.99 | — | — | 0.0014 |
| | | 10⁶ | 10⁻⁶ | | 10⁵ | 10⁻⁷ | | | 10⁻¹⁰ | | | | |
| | 7 | 8.63 × | 4.64 × | 0.63 | 1.01 × | 8.76 × | 0.48 | 20539 | 1.00 × | 0.93 | — | — | 0.0006 |
| | | 10⁵ | 10⁻⁶ | | 10⁵ | 10⁻⁷ | | | 10⁻⁹ | | | | |

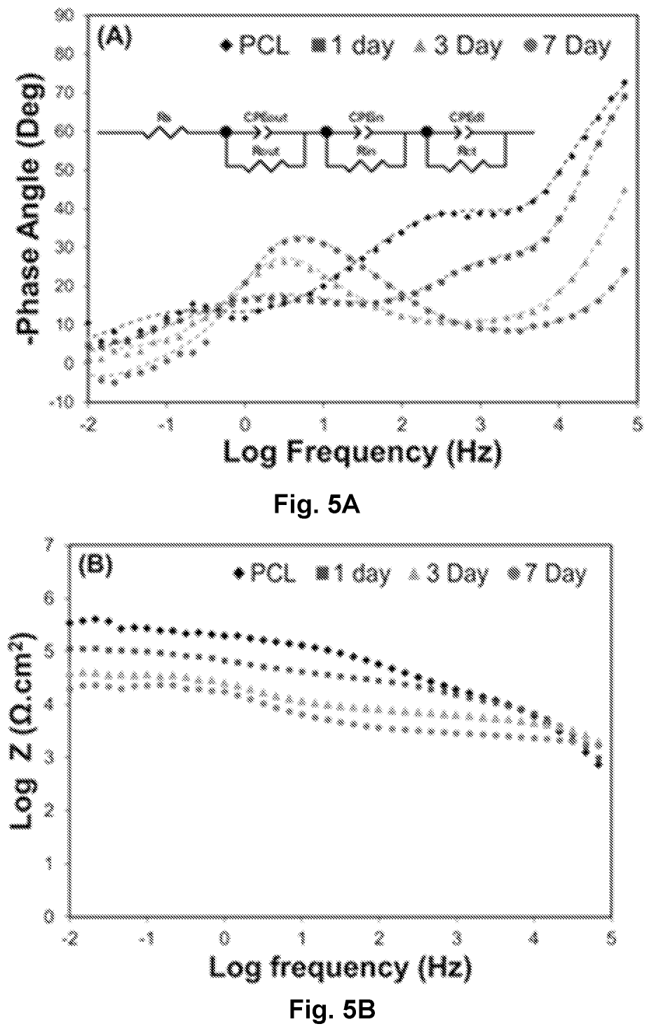

EIS, as a non-destructive technique, can allow us to continuously monitor the corrosion resistance properties of a sample over a long period, as it does not perturb the system significantly due to its steady state measurement [72]. The effect of lawsone inhibitor on long-term protection performance of the coatings was studied through EIS measurements by comparing barrier properties of PCL and PCL-LS coatings during a 7-day immersion period in Hank's solution. Bode plots of PCL and PCL-LS coatings after 1,3, and 7 days of immersion and the corresponding fitted EIS parameters are presented in FIG. 5A-D and Table 4, respectively. As can be seen in FIG. 5A, the phase angle of PCL at the high-frequency region started to sharply decline with increasing the immersion time, indicating diffusion of water and corrosive species into the PCL film and thus, rapid deterioration of barrier properties [73]. As immersion continued, a broad time constant in the medium-frequency range became more visible (at about 1-10 Hz), which is attributed to two combined time constants belong to the pore resistance of the coating and the oxide layer on the Mg alloy [66]. Moreover, an inductive loop appeared on the PCL spectra (at about 10⁻² Hz), which is attributed to the pitting corrosion and adsorption/desorption of intermediates on the metal substrate during immersion [72]. The Bode plot of the PCL-LS (FIG. 5C) showed a completely different trend. In contrast to the PCL, the phase angles of PCL-LS coating at the high-frequency region remained quite stable with much less variation during the immersion period. Besides, no inductive loop was observed in the PCL-LS spectra even after 7 days of immersion, proving the presence of a stable corrosion protective layer on the metal surface [74].

Variation of log |Z|_{0.01 Hz} for the PCL and PCL-LS over immersion time can be seen from Bode-impedance plots, which reflect the overall corrosion resistance of coatings. For the PCL coating (FIG. 5B), the |Z|_{0.01 Hz} value continued to decrease over 7 days of immersion test. With the increase of immersion time, PCL coating started to gradually lose its barrier properties due to the penetration of corrosive electrolyte into the coatings, resulting in a steady decrease of |Z|_{0.01 Hz} during the test [51]. It should be mentioned that the final |Z|_{0.01 Hz} value was still higher compared to uncoated Mg samples at the end of the immersion test. In contrast to PCL coating, PCL-LS (FIG. 5D) could maintain stable corrosion resistance and showed a better performance with a much lower reduction in |Z|_{0.1 Hz} values during 7 days of immersion. The results suggest that the lawsone inhibitor could effectively mitigate the development of corrosion and provide durable protection with long-term stability.

According to Table 4, the R_{ct}, Rₒᵤₜ, and Rᵢₙ values of PCL coatings all declined during the whole immersion time, reveling the successive deterioration of the physical barrier properties of the pure PCL coating over time [68]. In the case of PCL-LS coating, Rₒᵤₜ increased from 2.48×10⁶ to 6.84×10⁶ Ω.cm² after 24 h of immersion. Moreover, as the exposure time prolonged, n values related to CPE_{dl} continuously rose, indicating that the Mg alloy surface is becoming smoother by time. That is most likely due to the formation of insoluble metal-lawsone complexes upon the release of lawsone and filling of the active corroding sites on the Mg substrate [75]. It should be also noted that, at the end of 7 day immersion time, the R_{ct} value of PCL-LS coating is much higher than those of uncoated Mg samples and the pure PCL coating.

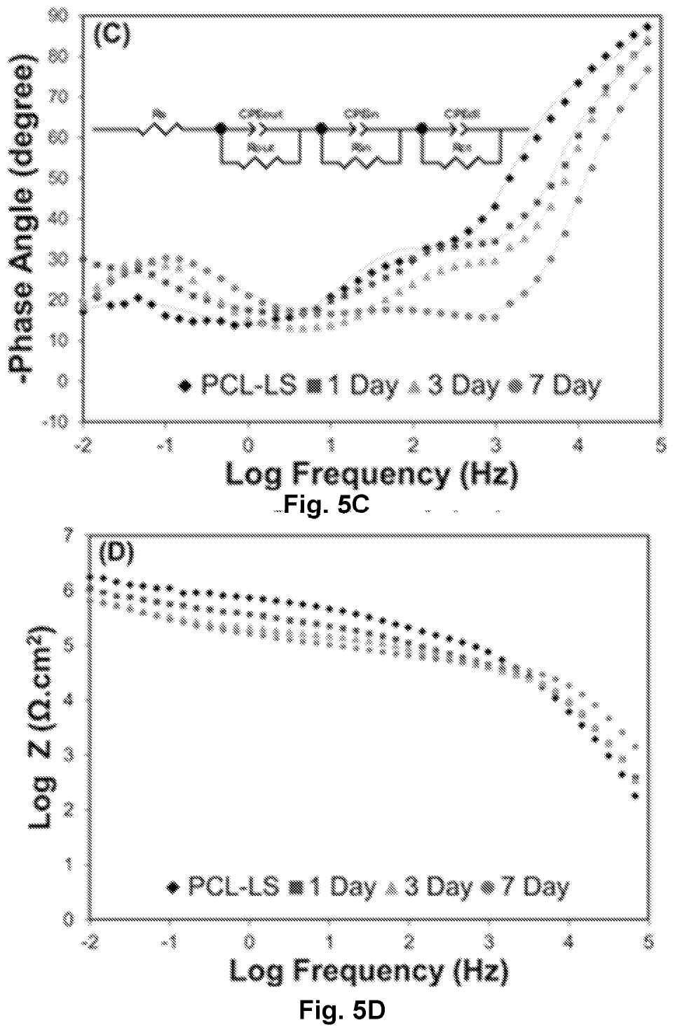

Immersion Corrosion Tests—FIG. 6 exhibits the post-corrosion SEM surface morphologies of Mg samples. After being immersed in Hank's solution for 7 days, the uncoated AZ31 and AZ31-OH samples displayed highly cracked surfaces with the presence of accumulated corrosion products on them, indicating that both samples underwent severe corrosion during the immersion test. Similarly, a high amount of corrosion products was observed on the PCL surface at the end of the immersion period, showing an insufficient corrosion protection performance by the pure PCL coating on the Mg alloy. Remarkably, the PCL-LS surface remained almost intact after 7 days of immersion and showed the least sign of corrosion with very minor defects on the polymer coating. The SEM observations suggested that the presence of lawsone mitigates the corrosion damages of the PCL coating during immersion and improved the barrier properties against the penetration of water and corrosive specious through the coating.

To further evaluate the long-term degradation of Mg samples, in vitro immersion studies were performed as a reliable measurement in addition to the typical electrochemical corrosion tests [76]. Corrosion of Mg alloys in aqueous electrolytes co-occurs with the generation of hydrogen gas and increment of pH value as per the below equation [77]:

Mg+2H₂O→Mg²⁺+2OH⁻+H₂ (g)

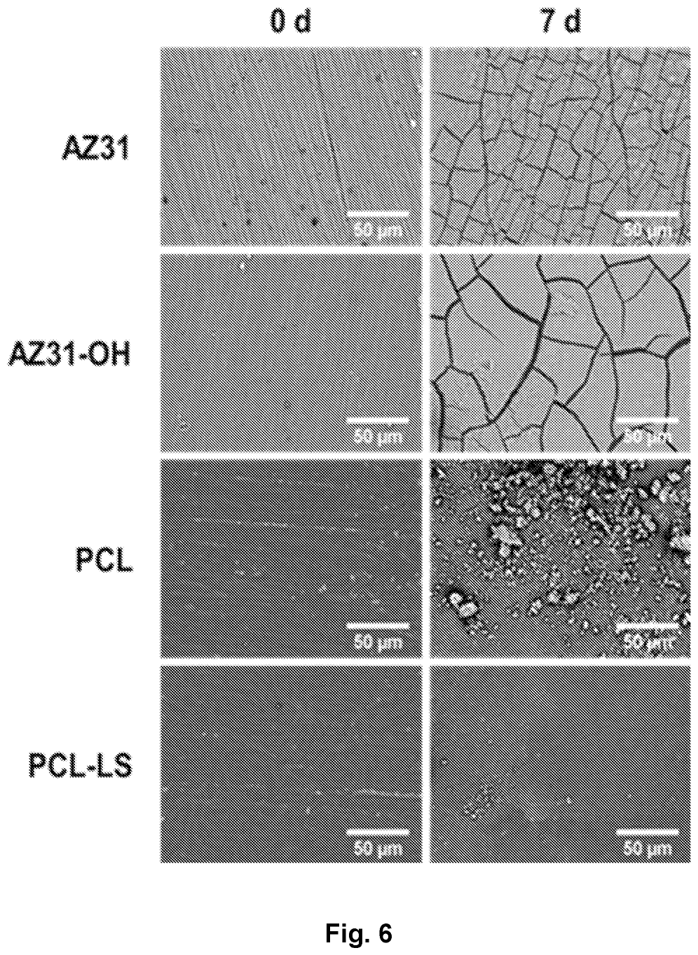

The corrosion of each mole of Mg atom results in the generation of one mole of hydrogen gas and two moles of OH⁻ anions. Therefore, monitoring the hydrogen evolution and pH value variation can provide precise information about the Mg corrosion as well as the protection efficiency of a coating on the Mg surface. FIG. 7A-B presents the variation in the hydrogen evolution volume and pH value during the immersion period of all experimental samples. As shown in FIG. 7A, the pristine AZ31 substrate showed a quick increase in the volume of evolved hydrogen and culminated to 7.92±0.67 mL.cm⁻² after 7 days of immersion, showing a very high reactivity and degradation rate of bare AZ31. For the AZ31-OH sample, the average of H₂ evolution rate within the first two days (0.41±0.19 mL.cm⁻².day⁻¹) was much slower than that of the pristine AZ31 substrate (1.20±0.17 mL.cm⁻².day⁻¹). After 2 days, it started to increase, and the evolved hydrogen reached a final volume of 6.10±0.84 mL.cm⁻² at the end of 7-day immersion. The slower rate at the initial stages is attributed to the presence of the protective Mg(OH)₂ layer resulted from alkaline treatment. However, in the presence of chloride ions, this layer will be converted into MgCl₂, which is less stable in aqueous solutions than Mg(OH)₂, increasing the corrosion rate [14]. PCL displayed a total hydrogen evolution volume of 3.025±0.31 mL.cm⁻² after 7 days, which was much lower compared to bare AZ31 and AZ31-OH samples. This implies the PCL coating can provide steady corrosion protection by acting as a barrier layer to inhibit the water/electrolyte diffusing into the substrate. The accumulated hydrogen volume of PCL-LS further decreased to 1.25±0.28 mL.cm⁻² in, showing the highest corrosion protection of PCL-LS coating among all tested samples.

The trend of pH changes (FIG. 7B) was consistent with the hydrogen evolution results. The AZ31 exhibited a sharp rise in pH from about 7.4 to above 8.8 within the first day of immersion and reached to 10.88±0.19 at the end. Although being successful in suppressing hydrogen generation at initial stages, the AZ31-OH sample did not show noticeably lower pH values than the AZ31 over the whole testing period. This might be due to the dissolution of the passive Mg(OH)₂ layer into the media, resulting in OH⁻ release and increase in pH values [78]. The pH values of the PCL and PCL-LS at the end of the immersion period were 8.57±0.26 and 8.16±0.10, respectively, which are much lower than that of uncoated Mg samples. It indicates that these coated samples were stable and did not undergo severe corrosion during immersion. Overall, the results of hydrogen evolution and pH change tests demonstrated that the corrosion resistance of Mg alloy was effectively improved by PCL coating and more significantly after incorporation of lawsone into the coating formulation.

ATR-FTIR was used to investigate the compositions of corrosion products after immersion for 7 days in Hank's solution. As displayed in FIG. 7C, the characteristic peaks for PO₄³⁻ appeared in the spectra of all samples at 991 cm⁻¹, indicating that the corrosion products are mainly phosphate [79, 80]. The intensities of these peaks are less pronounced in the alkaline treated and coated Mg samples, indicating the better anti-corrosion properties of them compared to AZ31. Moreover, the presence of sharp characteristic peaks at 1723 cm⁻¹ confirmed the existence of PCL coatings on the Mg samples after 7 days of immersion. It is worth mentioning that the intensity of the characteristic peak was higher in PCL-LS sample compared to the pure PCL coating. This indicates that in the absence of lawsone and its corrosion inhibition capability, the PCL coating degrades more rapidly within the immersion period.

Mechanism of Corrosion Inhibition of PCL-LS Coating—The corrosion inhibition property of the PCL-LS coating was verified by electrochemical corrosion studies as well as in vitro immersion tests. Lawsone is initially entrapped within the inner layer of the coating as the corrosion inhibitor, while a top PCL layer is coated on that as a barrier layer to minimize the leaching of the inhibitor. Once a defect appears within the PCL coating, the lawsone molecule is released from the inner layer into the damaged area. The anticorrosive properties of lawsone mainly rely on its ability to chelate metal cations—in our case Mg²⁺—to form a protective barrier on top of the underlying Mg substrate [38]. Exposing to a saline electrolyte like Hank's solution, the lawsone molecule undergoes a chemical rearrangement by delocalization of a pair of electrons on its hydroxyl group and then can form a complex with Mg²⁺ ions [81]. These insoluble complexes produced by lawsone molecules will be adsorbed onto the damaged area of the Mg surface and block the active sites of corrosion. The formed organic layer can seal and isolate the damaged substrate form the corrosive electrolyte and thus prevents further corrosion at the metal surface [36].

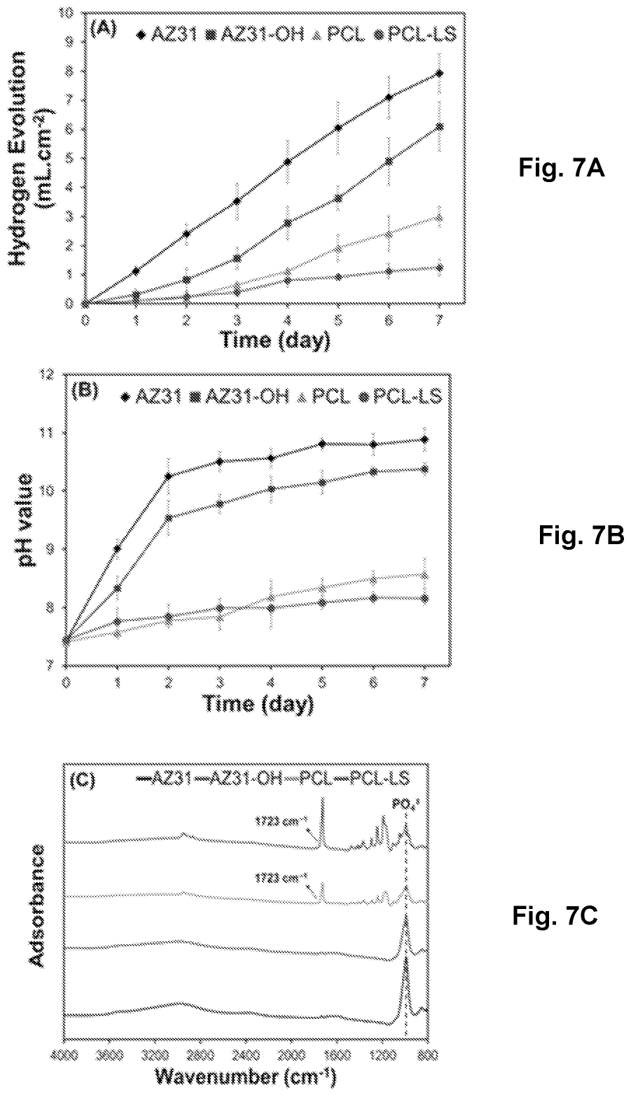

Antibacterial Studies—Antibacterial properties of the coatings were evaluated against E. coli and S. aureus, two bacterial strains mainly associated with the biomedical implant-related infections [44]. FIG. 8 shows the results of the disc diffusion test after 24 h of incubation at 37° C. As can be seen, no zone of inhibition was observed around AZ31, AZ31-OH, and PCL samples on agar plates, implying that these samples do not possess any antibacterial effects. In contrast, PCL-LS displayed clear zones of inhibition against E. coli and S. aureus with diameters of 26±0.5 mm and 22±0.8 mm, respectively. The ability of lawsone to inhibit bacterial growth has been reported previously in several studies [41, 43]. The bactericidal property of lawsone arises from its molecular structure (2-hydroxy-1,4 napthoquinone). The highly reactive quinone groups of lawsone interact with the nucleophilic amino acids of surface proteins and polypeptides present on the bacterial cell membrane and inactivate them by disturbing their functionalities, therefore induce antibacterial properties [82, 83]. The results suggest that the PCL-LS composite coating has a great potential for Mg alloys, as it offers both corrosion-protective and antibacterial functions.

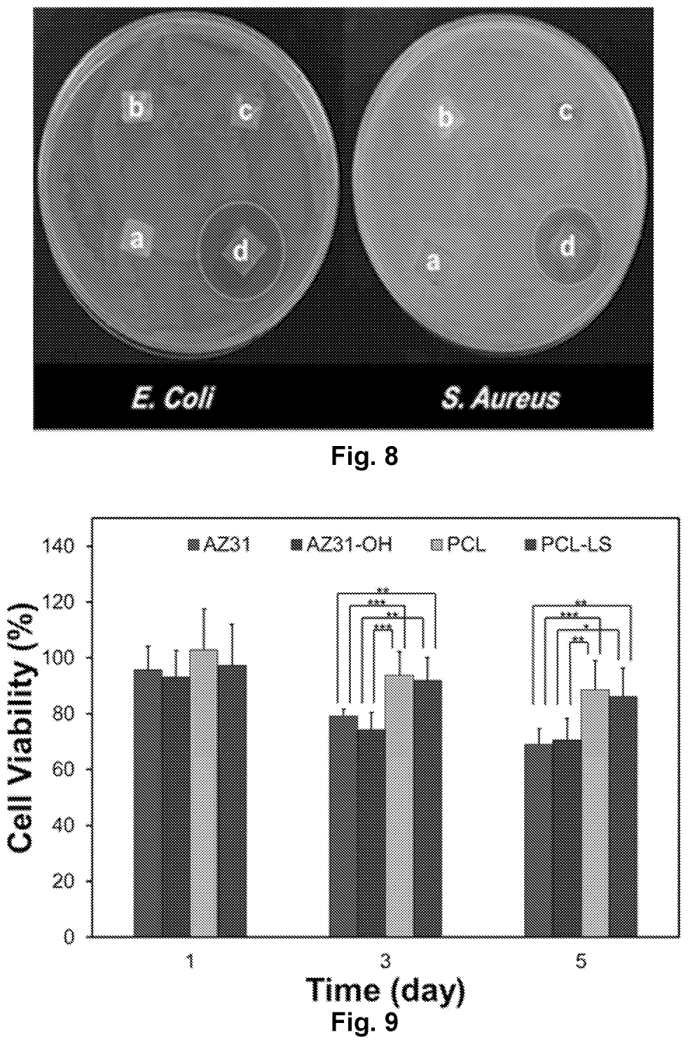

In vitro Cytocompatibility—To investigate the biosafety of coatings for bio-implant applications, the in vitro cytocompatibility of Mg samples were examined toward hFOB using CCK-8 test. FIG. 9 shows the viability percentage of hFOBs exposed to different Mg samples extractions for 1, 3, and 5 days. No significant difference was observed among the viability of samples after 1 day of incubation (P-value>0.5). However, as the incubation time prolonged, the viability of coated samples was significantly higher (P-value<0.5) than those of uncoated samples at 3-day and 5-day time intervals, indicating the improved cytocompatibility of the Mg substrates after applying the coatings. It should be mentioned that the viability values of both PCL and PCL-LS were 88.6±8.2% and 86.1±10.2% after 5 days of incubation, respectively. This demonstrates the acceptable cytocompatibility of the coatings, which is a primary requirement for bio-implant application [44]. The enhanced cell viability of coated samples can be attributed to their lower degradation rate [84]. It has been reported that the rapid degradation of bare Mg alloys leads to the formation of air gas pockets along with an increase in pH value of the culture environment. This will result in the altered cellular osmolality and damage of the cell membranes, which ultimately lead to cell death [85]. The cytocompatibility results indicated that applying PCL coatings promoted the cell viability on AZ31 substrates. Moreover, incorporation of lawsone as corrosion inhibitor into the PCL coating not only did not compromise the cytocompatibility but also remarkably improved the corrosion properties of the pure PCL coating and endowed it with a remarkable antibacterial property.

### Conclusion

In this example, lawsone was used as a natural corrosion inhibitor and incorporated into the biocompatible PCL polymeric coating to improve the corrosion resistance of biodegradable AZ31 Mg alloy. The results of electrochemical corrosion tests and in vitro immersion studies clearly indicated that the corrosion protection performance of the PCL coating was significantly enhanced after incorporation of lawsone into the coatings. Also, the corrosion resistance of the AZ31 substrate was increased by almost two order of magnitudes (IE of 98.3%) after being coated with PCL-LS. Lawsone-containing coatings showed strong antibacterial activity against E. coli and S. aureus, which can be highly beneficial for bio-implant applications as it minimizes the risk of implant-related infection at the early stages. While most of commonly used Mg corrosion inhibitors have limited biomedical applications due to the concerns over their cytocompatibility, no cytotoxic effect was observed for PCL-LS coating toward hFOB cells. The results of this study suggested the proposed polymeric coating based on lawsone has great potential as a protective coating to expand the applicability of Mg alloys for biodegradable implant applications.

### EXAMPLE 1 REFERENCES

[1] D. Zhao, F. Witte, F. Lu, J. Wang, J. Li, and L. Qin, “Current status on clinical applications of magnesium-based orthopaedic implants: A review from clinical translational perspective,” Biomaterials, vol. 112, pp. 287-302, 2017.
[2] S. Jiang et al., “Effect of alkali/acid pretreatment on the topography and corrosion resistance of as-deposited CaP coating on magnesium alloys,” Journal of Alloys and Compounds, vol. 793, pp. 202-211, 2019.
[3] M. P. Staiger, A. M. Pietak, J. Huadmai, and G. Dias, “Magnesium and its alloys as orthopedic biomaterials: a review,” Biomaterials, vol. 27, no. 9, pp. 1728-1734, 2006.
[4] Y. Xin, T. Hu, and P. Chu, “In vitro studies of biomedical magnesium alloys in a simulated physiological environment: a review,” Acta biomaterialia, vol. 7, no. 4, pp. 1452-1459, 2011.
[5] Y. Chen, Z. Xu, C. Smith, and J. Sankar, “Recent advances on the development of magnesium alloys for biodegradable implants,” Acta biomaterialia, vol. 10, no. 11, pp. 4561-4573, 2014.
[6] W. Zhang et al., “Strengthened corrosion control of poly (lactic acid) (PLA) and poly (ε-caprolactone) (PCL) polymer-coated magnesium by imbedded hydrophobic stearic acid (SA) thin layer,” Corrosion Science, vol. 112, pp. 327-337, 2016.
[7] P. Xiong et al., “A pH-sensitive self-healing coating for biodegradable magnesium implants,” Acta biomaterialia, vol. 98, pp. 160-173, 2019.
[8] A. Zomorodian et al., “Anti-corrosion performance of a new silane coating for corrosion protection of AZ31 magnesium alloy in Hank's solution,” Surface and coatings technology, vol. 206, no. 21, pp. 4368-4375, 2012.
[9] T. Da Conceicao, N. Scharnagl, C. Blawert, W. Dietzel, and K. Kainer, “Surface modification of magnesium alloy AZ31 by hydrofluoric acid treatment and its effect on the corrosion behaviour,” Thin Solid Films, vol. 518, no. 18, pp. 5209-5218, 2010.
[10] H. Chai et al., “In vitro and in vivo evaluations on osteogenesis and biodegradability of a β-tricalcium phosphate coated magnesium alloy,” Journal of Biomedical Materials Research Part A, vol. 100, no. 2, pp. 293-304, 2012.
[11] A. F. Cipriano et al., “Anodization of magnesium for biomedical applications—Processing, characterization, degradation and cytocompatibility,” Acta biomaterialia, vol. 62, pp. 397-417, 2017.
[12] X.-j. Cui, X.-z. Lin, C.-h. Liu, R.-s. Yang, X.-w. Zheng, and M. Gong, “Fabrication and corrosion resistance of a hydrophobic micro-arc oxidation coating on AZ31 Mg alloy,” Corrosion Science, vol. 90, pp. 402-412, 2015.
[13] L.-Y. Li et al., “Advances in functionalized polymer coatings on biodegradable magnesium alloys-a review,” Acta biomaterialia, vol. 79, pp. 23-36, 2018.
[14] A. Zomorodian, M. Garcia, T. M. e Silva, J. Fernandes, M. Fernandes, and M. Montemor, “Corrosion resistance of a composite polymeric coating applied on biodegradable AZ31 magnesium alloy,” Acta Biomaterialia, vol. 9, no. 10, pp. 8660-8670, 2013.
[15] P. Shi, B. Niu, E. Shanshan, Y. Chen, and Q. Li, “Preparation and characterization of PLA coating and PLA/MAO composite coatings on AZ31 magnesium alloy for improvement of corrosion resistance,” Surface and Coatings Technology, vol. 262, pp. 26-32, 2015.
[16] J. Li, P. Cao, X. Zhang, S. Zhang, and Y. He, “In vitro degradation and cell attachment of a PLGA coated biodegradable Mg-6Zn based alloy,” Journal of materials science, vol. 45, no. 22, pp. 6038-6045, 2010.
[17] H. Li, F. Peng, D. Wang, Y. Qiao, D. Xu, and X. Liu, “Layered double hydroxide/poly-dopamine composite coating with surface heparinization on Mg alloys: improved anticorrosion, endothelialization and hemocompatibility,” Biomaterials science, vol. 6, no. 7, pp. 1846-1858, 2018.
[18] J. Kim, H. M. Mousa, C. H. Park, and C. S. Kim, “Enhanced corrosion resistance and biocompatibility of AZ31 Mg alloy using PCL/ZnO NPs via electrospinning,” Applied Surface Science, vol. 396, pp. 249-258, 2017.
[19] X. Gu et al., “Surface modification of an Mg-1Ca alloy to slow down its biocorrosion by chitosan,” Biomedical Materials, vol. 4, no. 4, p. 044109, 2009.
[20] S. Kunjukunju, A. Roy, M. Ramanathan, B. Lee, J. E. Candiello, and P. N. Kumta, “A layer-by-layer approach to natural polymer-derived bioactive coatings on magnesium alloys,” Acta biomaterialia, vol. 9, no. 10, pp. 8690-8703, 2013.
[21] P. Xiong et al., “Osteogenic and pH stimuli-responsive self-healing coating on biomedical Mg-1Ca alloy,” Acta biomaterialia, vol. 92, pp. 336-350, 2019.
[22] X. Wang et al., “Active corrosion protection of super-hydrophobic corrosion inhibitor intercalated Mg—Al layered double hydroxide coating on AZ31 magnesium alloy,” Journal of Magnesium and Alloys, 2020.
[23] S. Abbaspoor, A. Ashrafi, and R. Abolfarsi, “Development of self-healing coatings based on ethyl cellulose micro/nano-capsules,” Surface Engineering, vol. 35, no. 3, pp. 273-280, 2019.
[24] S. Garcia, H. Fischer, and S. Van Der Zwaag, “A critical appraisal of the potential of self healing polymeric coatings,” Progress in Organic Coatings, vol. 72, no. 3, pp. 211-221, 2011.
[25] L. M. Calado, M. G. Taryba, Y. Morozov, M. J. Carmezim, and M. F. Montemor, “Novel Smart and Self-Healing Cerium Phosphate-based Corrosion Inhibitor for AZ31 Magnesium Alloy,” Corrosion Science, p. 108648, 2020.
[26] M. J. Anjum et al., “In-situ intercalation of 8-hydroxyquinoline in Mg—Al LDH coating to improve the corrosion resistance of AZ31,” Corrosion Science, vol. 157, pp. 1-10, 2019.
[27] G. Zhang et al., “A novel approach to fabricate protective layered double hydroxide films on the surface of anodized Mg—Al alloy,” Advanced Materials Interfaces, vol. 4, no. 12, p. 1700163, 2017.
[28] R.-C. Zeng, Z.-G. Liu, F. Zhang, S.-Q. Li, H.-Z. Cui, and E.-H. Han, “Corrosion of molybdate intercalated hydrotalcite coating on AZ31 Mg alloy,” Journal of Materials Chemistry A, vol. 2, no. 32, pp. 13049-13057, 2014.
[29] M. Sun, A. Yerokhin, M. Y. Bychkova, D. Shtansky, E. Levashov, and A. Matthews, “Self-healing plasma electrolytic oxidation coatings doped with benzotriazole loaded halloysite nanotubes on AM50 magnesium alloy,” Corrosion Science, vol. 111, pp. 753-769, 2016.
[30] S. A. Umoren, M. M. Solomon, A. Madhankumar, and I. B. Obot, “Exploration of natural polymers for use as green corrosion inhibitors for AZ31 magnesium alloy in saline environment,” Carbohydrate Polymers, vol. 230, p. 115466, 2020.
[31] W. Shang, C. He, Y. Wen, Y. Wang, and Z. Zhang, “Performance evaluation of triethanolamine as corrosion inhibitor for magnesium alloy in 3.5 wt % NaCl solution,” RSC advances, vol. 6, no. 115, pp. 113967-113980, 2016.
[32] E. E. Oguzie, “Evaluation of the inhibitive effect of some plant extracts on the acid corrosion of mild steel,” Corrosion science, vol. 50, no. 11, pp. 2993-2998, 2008.
[33] S. Mo, H.-Q. Luo, and N.-B. Li, “Plant extracts as “green” corrosion inhibitors for steel in sulphuric acid,” Chemical Papers, vol. 70, no. 9, pp. 1131-1143, 2016.
[34] A. Abdel-Gaber, B. Abd-El-Nabey, I. Sidahmed, A. EI-Zayady, and M. Saadawy, “Inhibitive action of some plant extracts on the corrosion of steel in acidic media,” Corrosion science, vol. 48, no. 9, pp. 2765-2779, 2006.
[35] S. Dananjaya, M. Edussuriya, and A. Dissanayake, “Inhibition action of lawsone on the corrosion of mild steel in acidic media,” The Online Journal of Science and Technology, vol. 2, no. 2, pp. 32-36, 2012.
[36] A. Ostovari, S. Hoseinieh, M. Peikari, S. Shadizadeh, and S. Hashemi, “Corrosion inhibition of mild steel in 1 M HCl solution by henna extract: A comparative study of the inhibition by henna and its constituents (Lawsone, Gallic acid, α-d-Glucose and Tannic acid),” Corrosion Science, vol. 51, no. 9, pp. 1935-1949, 2009.
[37] A. Motalebi, M. Nasr-Esfahani, R. Ali, and M. Pourriahi, “Improvement of corrosion performance of 316L stainless steel via PVTMS/henna thin film,” Progress in Natural Science: Materials International, vol. 22, no. 5, pp. 392-400, 2012.
[38] M. Odarczenko et al., “Self-protecting epoxy coatings with anticorrosion microcapsules,” ACS omega, vol. 3, no. 10, pp. 14157-14164, 2018.
[39] S. Lamaka, B. Vaghefinazari, D. Mei, R. Petrauskas, D. Hoche, and M. Zheludkevich, “Comprehensive screening of Mg corrosion inhibitors,” Corrosion Science, vol. 128, pp. 224-240, 2017.
[40] B. S. Nayak, G. Isitor, E. Davis, and G. Pillai, “The evidence based wound healing activity of Lawsonia inermis Linn,” Phytotherapy Research: An International Journal Devoted to Pharmacological and Toxicological Evaluation of Natural Product Derivatives, vol. 21, no. 9, pp. 827-831, 2007.
[41] M. A. Ur Rehman et al., “Electrophoretic deposition of lawsone loaded bioactive glass (BG)/chitosan composite on polyetheretherketone (PEEK)/BG layers as antibacterial and bioactive coating,” Journal of Biomedical Materials Research Part A, vol. 106, no. 12, pp. 3111-3122, 2018.
[42] M. Barani, M. Mirzaei, M. Torkzadeh-Mahani, and M. H. Nematollahi, “Lawsone-loaded Niosome and its antitumor activity in MCF-7 breast Cancer cell line: a Nano-herbal treatment for Cancer,” DARU Journal of Pharmaceutical Sciences, vol. 26, no. 1, pp. 11-17, 2018.
[43] M. Adeli-Sardou, M. M. Yaghoobi, M. Torkzadeh-Mahani, and M. Dodel, “Controlled release of lawsone from polycaprolactone/gelatin electrospun nano fibers for skin tissue regeneration,” International journal of biological macromolecules, vol. 124, pp. 478-491, 2019.
[44] Y.-H. Zou et al., “Corrosion resistance and antibacterial activity of zinc-loaded montmorillonite coatings on biodegradable magnesium alloy AZ31,” Acta biomaterialia, vol. 98, pp. 196-214, 2019.
[45] L. Zhang, J. Zhang, C.-f. Chen, and Y. Gu, “Advances in microarc oxidation coated AZ31 Mg alloys for biomedical applications,” Corrosion Science, vol. 91, pp. 7-28, 2015.
[46] S. Zhang et al., “Research on an Mg—Zn alloy as a degradable biomaterial,” Acta biomaterialia, vol. 6, no. 2, pp. 626-640, 2010.
[47] C.-J. Pan et al., “Effects of self-assembly of 3-phosphonopropionic acid, 3-aminopropyltrimethoxysilane and dopamine on the corrosion behaviors and biocompatibility of a magnesium alloy,” Materials Science and Engineering: C, vol. 67, pp. 132-143, 2016.
[48] J. B. Choi et al., “Effect of composite coating with poly-dopamine/PCL on the corrosion resistance of magnesium,” International Journal of Polymeric Materials and Polymeric Biomaterials, vol. 68, no. 6, pp. 328-337, 2019.
[49] H. Bakhsheshi-Rad et al., “Synthesis and corrosion behavior of a hybrid bioceramic-biopolymer coating on biodegradable Mg alloy for orthopaedic implants,” Journal of Alloys and Compounds, vol. 648, pp. 1067-1071, 2015.
[50] P. Xiong et al., “Biomimetic Ca, Sr/P-doped silk fibroin films on Mg-1Ca alloy with dramatic corrosion resistance and osteogenic activities,” ACS Biomaterials Science & Engineering, vol. 4, no. 9, pp. 3163-3176, 2018.
[51] A. Mohammadi, M. Barikani, A. H. Doctorsafaei, A. P. Isfahani, E. Shams, and B. Ghalei, “Aqueous dispersion of polyurethane nanocomposites based on calix [4] arenes modified graphene oxide nanosheets: preparation, characterization, and anti-corrosion properties,” Chemical Engineering Journal, vol. 349, pp. 466-480, 2018.
[52] J. Hudzicki, “Kirby-Bauer disk diffusion susceptibility test protocol,” 2009.
[53] P. Wang, P. Xiong, J. Liu, S. Gao, T. Xi, and Y. Cheng, “A silk-based coating containing GREDVY peptide and heparin on Mg—Zn—Y—Nd alloy: improved corrosion resistance, hemocompatibility and endothelialization,” Journal of Materials Chemistry B, vol. 6, no. 6, pp. 966-978, 2018.
[54] C.-J. Pan et al., “Improving corrosion resistance and biocompatibility of magnesium alloy by sodium hydroxide and hydrofluoric acid treatments,” Applied Sciences, vol. 7, no. 1, p. 33, 2017.
[55] A. I. Visan et al., “Long-term evaluation of dip-coated pcl-blend-peg coatings in simulated conditions,” Polymers, vol. 12, no. 3, p. 717, 2020.
[56] H. Liu and T. J. Webster, “Nanomedicine for implants: a review of studies and necessary experimental tools,” Biomaterials, vol. 28, no. 2, pp. 354-369, 2007.
[57] S. Agarwal et al., “Enhanced corrosion protection and biocompatibility of a PLGA-silane coating on AZ31 Mg alloy for orthopaedic applications,” RSC advances, vol. 6, no. 115, pp. 113871-113883, 2016.
[58] G. Song, A. Atrens, D. St John, X. Wu, and J. Nairn, “The anodic dissolution of magnesium in chloride and sulphate solutions,” Corrosion science, vol. 39, no. 10-11, pp. 1981-2004, 1997.
[59] N. Eliaz, “Corrosion of metallic biomaterials: A review,” Materials, vol. 12, no. 3, p. 407, 2019.
[60] V. Shkirskiy et al., “Revisiting the electrochemical impedance spectroscopy of magnesium with online inductively coupled plasma atomic emission spectroscopy,” Chem Phys Chem, vol. 16, no. 3, pp. 536-539, 2015.
[61] B. M. Shafiee, R. Torkaman, M. Mahmoudi, and R. Emadi, “An improvement in corrosion resistance of 316L AISI coated using PCL-gelatin composite by dip-coating method,” Progress in Organic Coatings, vol. 130, pp. 200-205, 2019.
[62] D. P. Bhattarai, S. Shrestha, B. K. Shrestha, C. H. Park, and C. S. Kim, “A controlled surface geometry of polyaniline doped titania nanotubes biointerface for accelerating MC3T3-E1 cells growth in bone tissue engineering,” Chemical Engineering Journal, vol. 350, pp. 57-68, 2018.
[63] D. A. Leal, I. C. Riegel-Vidotti, M. G. S. Ferreira, and C. E. B. Marino, “Smart coating based on double stimuli-responsive microcapsules containing linseed oil and benzotriazole for active corrosion protection,” Corrosion Science, vol. 130, pp. 56-63, 2018.
[64] X. He et al., “Biocompatibility, corrosion resistance and antibacterial activity of TiO2/CuO coating on titanium,” Ceramics International, vol. 43, no. 18, pp. 16185-16195, 2017.
[65] H. Qian et al., “Dual-action smart coatings with a self-healing superhydrophobic surface and anti-corrosion properties,” Journal of materials chemistry A, vol. 5, no. 5, pp. 2355-2364, 2017.
[66] Y. Huang et al., “Triple-action self-healing protective coatings based on shape memory polymers containing dual-function microspheres,” ACS applied materials & interfaces, vol. 10, no. 27, pp. 23369-23379, 2018.
[67] J. Li, J. Cui, J. Yang, Y. Li, H. Qiu, and J. Yang, “Reinforcement of graphene and its derivatives on the anticorrosive properties of waterborne polyurethane coatings,” Composites Science and Technology, vol. 129, pp. 30-37, 2016.
[68] X. Liu, C. Gu, Z. Wen, and B. Hou, “Improvement of active corrosion protection of carbon steel by water-based epoxy coating with smart CeO2 nanocontainers,” Progress in Organic Coatings, vol. 115, pp. 195-204, 2018.
[69] B. Mingo, Y. Guo, R. Leiva-Garcia, B. Connolly, A. Matthews, and A. Yerokhin, “Smart Functionalization of Ceramic-coated AZ31 Magnesium Alloy,” ACS Applied Materials & Interfaces, 2020.
[70] M. Plawecka, D. Snihirova, B. Martins, K. Szczepanowicz, P. Warszynski, and M. Montemor, “Self healing ability of inhibitor-containing nanocapsules loaded in epoxy coatings applied on aluminium 5083 and galvanneal substrates,” Electrochimica Acta, vol. 140, pp. 282-293, 2014.
[71] M. T. Majd, T. Shahrabi, and B. Ramezanzadeh, “The role of neodymium based thin film on the epoxy/steel interfacial adhesion and corrosion protection promotion,” Applied Surface Science, vol. 464, pp. 516-533, 2019.
[72] S. Feliu, “Electrochemical Impedance Spectroscopy for the Measurement of the Corrosion Rate of Magnesium Alloys: Brief Review and Challenges,” Metals, vol. 10, no. 6, p. 775, 2020.
[73] M. Yeganeh, N. Asadi, M. Omidi, and M. Mandavian, “An investigation on the corrosion behavior of the epoxy coating embedded with mesoporous silica nanocontainer loaded by sulfamethazine inhibitor,” Progress in Organic Coatings, vol. 128, pp. 75-81, 2019.
[74] R. Arrabal et al., “Effect of Nd on the corrosion behaviour of AM50 and AZ91D magnesium alloys in 3.5 wt. % NaCl solution,” Corrosion Science, vol. 55, pp. 301-312, 2012.
[75] M. T. Majd, M. Davoudi, M. Ramezanzadeh, E. Ghasemi, B. Ramezanzadeh, and M. Mandavian, “Construction of a smart active/barrier anti-corrosion system based on epoxy-ester/zinc intercalated kaolin nanocontainer for steel substrate,” Construction and Building Materials, vol. 247, p. 118555, 2020.
[76] A. D. King, N. Birbilis, and J. R. Scully, “Accurate electrochemical measurement of magnesium corrosion rates; a combined impedance, mass-loss and hydrogen collection study,” Electrochimica Acta, vol. 121, pp. 394-406, 2014.
[77] E. Ghali, W. Dietzel, and K.-U. Kainer, “General and localized corrosion of magnesium alloys: a critical review,” Journal of materials engineering and performance, vol. 13, no. 1, pp. 7-23, 2004.
[78] S. Zhang et al., “In vitro degradation, hemolysis and MC3T3-E1 cell adhesion of biodegradable Mg—Zn alloy,” Materials Science and Engineering: C, vol. 29, no. 6, pp. 1907-1912, 2009.
[79] Y. Xin, K. Huo, T. Hu, G. Tang, and P. K. Chu, “Corrosion products on biomedical magnesium alloy soaked in simulated body fluids,” Journal of materials research, vol. 24, no. 8, pp. 2711-2719, 2009.
[80] B. Wang, D. Xu, J. Dong, and W. Ke, “Effect of corrosion product films on the in vitro degradation behavior of Mg-3% Al-1% Zn (in wt %) alloy in Hank's solution,” Journal of materials science & technology, vol. 34, no. 10, pp. 1756-1764, 2018.
[81] Y. Fang, B. Suganthan, and R. P. Ramasamy, “Electrochemical characterization of aromatic corrosion inhibitors from plant extracts,” Journal of Electroanalytical Chemistry, vol. 840, pp. 74-83, 2019.
[82] V. G. Dev, J. Venugopal, S. Sudha, G. Deepika, and S. Ramakrishna, “Dyeing and antimicrobial characteristics of chitosan treated wool fabrics with henna dye,” Carbohydrate Polymers, vol. 75, no. 4, pp. 646-650, 2009.
[83] N. Rahmoun, Z. Boucherit-Otmani, K. Boucherit, M. Benabdallah, D. Villemin, and N. Choukchou-Braham, “Antibacterial and antifungal activity of lawsone and novel naphthoquinone derivatives,” Medecine et maladies infectieuses, vol. 42, no. 6, pp. 270-275, 2012.
[84] T. S. Narayanan and M. H. Lee, “A simple strategy to modify the porous structure of plasma electrolytic oxidation coatings on magnesium,” RSC advances, vol. 6, no. 19, pp. 16100-16114, 2016.
[85] H. Bakhsheshi-Rad et al., “In vitro degradation behavior, antibacterial activity and cytotoxicity of TiO2-MAO/ZnHA composite coating on Mg alloy for orthopedic implants,” Surface and Coatings Technology, vol. 334, pp. 450-460, 2018.

### Example 2

Additional tests were performed to characterize the PCL-lawsone (PCL-LS) coating on AZ31 Mg alloy.

Coating adhesion test: Adhesion strength of the PCL-LS protective coating to the AZ31 substrate was examined by the cross-cut tape test according to ASTM D3359. A lattice of 100 squares of 1 mm² area each was formed on the coated samples using a sharp blade and a cross-cut guide. An adhesive tape (SEMicro CHT) was applied to the cross-cut area, rubbed with an eraser to ensure a firm contact between the tape and the test area, and quickly pulled off at an angle of 180° after 90 s. Depending on the percentage damage of the coatings, adhesion was graded according to ASTM standard chart, where 5B represents excellent adhesion (0% of coating detachment) and 0B represents very poor adhesion (>65% of coating detachment).

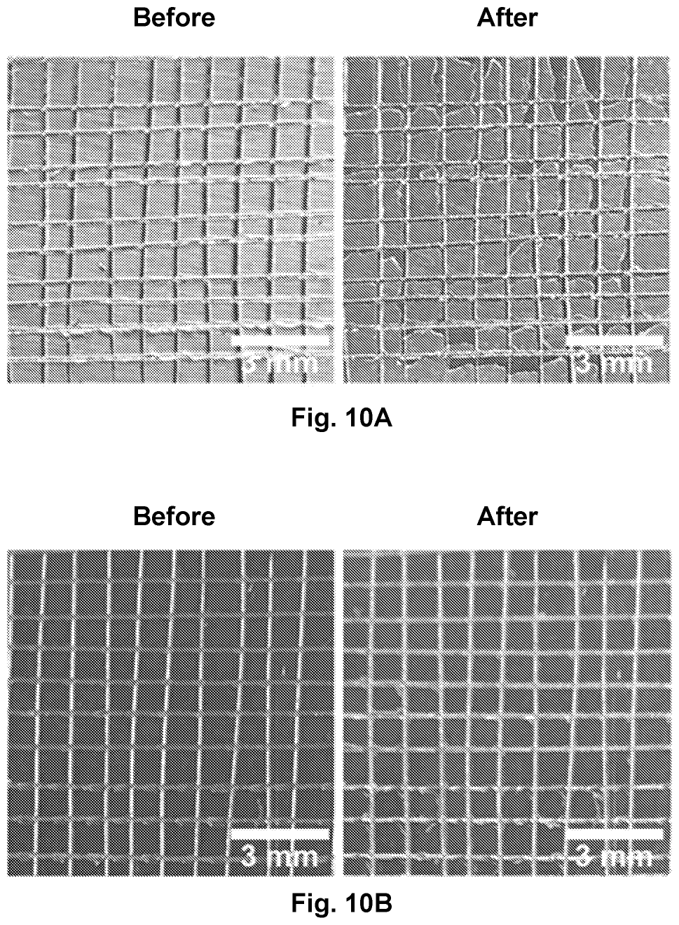

Having an adequate adhesion strength to the underlaying substrate is critical for any protective coatings as it determines the functionality and durability of the coating for the intended period of application. Interfacial adhesion between the Mg substrates and the PCL-LS coating was evaluated by the cross-cut adhesion test. FIGS. 10A and 10B show the surface of the PCL-LS coating on Mg alloy substrates before and after tape test. From the photographs after the tape removal, the adhesion strength of the PCL-LS to untreated AZ31 (FIG. 10A) and AZ31-OH (FIG. 10B) were classified as 3B and 4B, respectively. Compared to bare AZ31, AZ31-OH showed an improved adhesiveness to the PCL-LS coating. This is most likely due to the emerged numerous hydroxyl groups on the metal surface resulted from the alkaline pretreatment, which in turns can chemically interact with COOH groups of PCL and improve the coating/substrate interfacial adhesion. The results suggested that the PCL-LS coating has acceptable adhesion strength on the alkaline treated AZ31substrate for biomedical applications.

Cross-sectional SEM and EDS analyses: The surface and cross-section morphologies of the coatings were visualized by a scanning electron microscope (SEM, FEI Teneo, FEI Co.). The elemental analysis across the coating/substrate interface was obtained by an energy dispersive x-ray spectrometer (EDS) connected to the SEM.

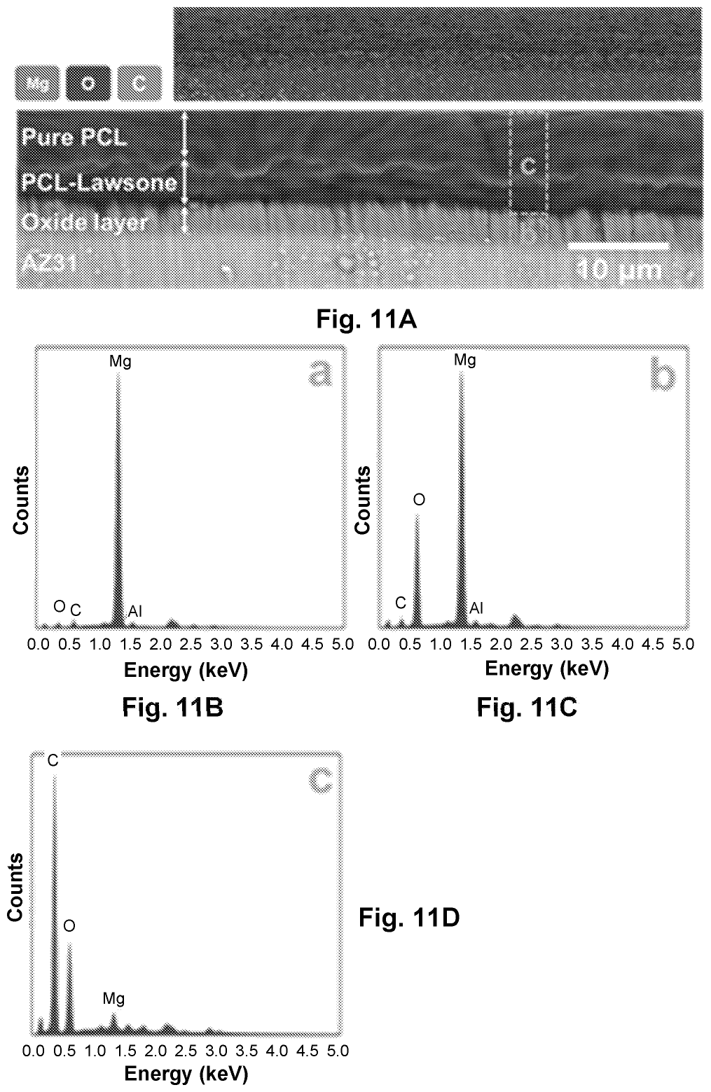

FIGS. 11A-11D show the cross-sectional morphology of the PCL-LS/substrate interface with the corresponding elemental analysis mapping and EDS spectra. The bi-layered structure of the polymeric coating composed of an inner PCL-lawsone and a top pure PCL layer along with the formed oxide layer on the AZ31 substrate surface were clearly observed (FIG. 11A). The PCL-LS coating was quite compact and uniformly covered the pretreated AZ31 substrate with no distinct gap across the interface, indicating the success in coating strategy. The thicknesses of the polymeric coating and the oxide layer were found to be about 10 μm and 4 μm, respectively. EDS analysis (FIGS. 11B-11D) was conducted to confirm the elemental composition of each layer. As expected, section A from the AZ31 substrate showed a sharp peak related to Mg element (FIG. 11B corresponds to Section a shown in FIG. 11A). In the EDS spectrum of section b, a new O peak was emerged, confirming the nature of oxide layer (FIG. 11C corresponds to Section b shown in FIG. 11A). Additionally, the highest content of C element was observed in Section c, corresponding to the PCL structure (FIG. 11D corresponds to Section c shown in FIG. 11A).

### Aspects of the Disclosure

The present disclosure will be better understood upon reading the following numbered aspects, which should not be confused with the claims. Any of the numbered aspects below can, in some instances, be combined with aspects described elsewhere in this disclosure and such combinations are intended to form part of the disclosure.

Aspect 1. A biodegradable orthopedic implant, comprising: an implant comprising a Mg alloy; and a coating comprising PCL and lawsone.

Aspect 2. The biodegradable orthopedic implant of aspect 1, wherein the coating comprises a first layer, a second layer, and a third layer; wherein the first layer is an oxide layer on an external surface of the implant; wherein the second layer comprises lawsone embedded in PCL; and wherein the third layer comprises a biocompatible polymer. In some aspects, the biocompatible polymer can comprise PCL.

Aspect 3. The biodegradable orthopedic implant of aspect 1 or 2, wherein the Mg alloy is a Mg—Al—Zn alloy.

Aspect 4. The biodegradable orthopedic implant of aspect 3, wherein the Mg—Al—Zn alloy has an Al content of about 3%.

Aspect 5. The biodegradable orthopedic implant of aspect 3, wherein the Mg—Al—Zn alloy is AZ31.

Aspect 6. The biodegradable orthopedic implant of any of aspects 2-5, wherein the second layer comprises about 0.1% to 1% w/w of lawsone to PCL.

Aspect 7. A corrosion-resistant coating for orthopedic implants, comprising: a first layer, a second layer, and a third layer; wherein the first layer is an oxide layer coated on an external surface of a magnesium alloy implant; wherein the second layer comprises lawsone embedded in PCL; and wherein the third layer comprises a biocompatible polymer. In some aspects, the biocompatible polymer can comprise PCL.

Aspect 8. The corrosion-resistant coating for orthopedic implants of aspect 7, wherein the second layer comprises about 0.1% to 1% w/w of lawsone to PCL.

Aspect 9. The corrosion-resistant coating for orthopedic implants of aspect 7 or 8, wherein the magnesium alloy implant comprises a Mg—Al—Zn alloy having an Al content of about 3%.

Aspect 10. A method of making a coated orthopedic implant, comprising immersing a magnesium alloy implant in an alkaline solution to form an oxide-coated implant; and coating the oxide-coated implant with a PCL-lawsone solution to form a coated orthopedic implant.

Aspect 11. The method of aspect 10, further comprising applying a protective polymer coating to the coated orthopedic implant. In some aspects, the protective polymer coating can comprise PCL.

Aspect 12. The method of aspect 10 or 11, wherein the alkaline solution is about 0.1-5 M NaOH.

Aspect 13. The method of any of aspects 10-12, wherein the PCL-lawsone solution comprises about 1% w/w of lawsone to PCL.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, “about 0” can refer to 0, 0.001, 0.01, or 0.1. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

What is claimed is:

1. A biodegradable orthopedic implant, comprising:
an implant comprising a Mg alloy; and
a coating comprising PCL and lawsone.

2. The biodegradable orthopedic implant of claim 1, wherein the coating comprises a first layer, a second layer, and a third layer;
wherein the first layer is an oxide layer on an external surface of the implant;
wherein the second layer comprises lawsone embedded in PCL; and
wherein the third layer comprises a biocompatible polymer.

3. The biodegradable orthopedic implant of claim 1, wherein the Mg alloy is a Mg—Al—Zn alloy.

4. The biodegradable orthopedic implant of claim 3, wherein the Mg—Al—Zn alloy has an Al content of about 3%.

5. The biodegradable orthopedic implant of claim 3, wherein the Mg—Al—Zn alloy is AZ31.

6. The biodegradable orthopedic implant of claim 2, wherein the second layer comprises about 0.1% to 1% w/w of lawsone to PCL.

7. The biodegradable orthopedic implant of claim 2, wherein the biocompatible polymer comprises PCL.

8. A corrosion-resistant coating for orthopedic implants, comprising:
a first layer, a second layer, and a third layer;
wherein the first layer is an oxide layer coated on an external surface of a magnesium alloy implant;
wherein the second layer comprises lawsone embedded in PCL; and
wherein the third layer comprises a biocompatible polymer.

9. The corrosion-resistant coating for orthopedic implants of claim 8, wherein the second layer comprises about 0.1% to 1% w/w of lawsone to PCL.

10. The corrosion-resistant coating for orthopedic implants of claim 8, wherein the magnesium alloy implant comprises a Mg—Al—Zn alloy having an Al content of about 3%.

11. The corrosion-resistant coating for orthopedic implants of claim 8, wherein the biocompatible polymer comprises PCL.

12. A method of making a coated orthopedic implant, comprising:
immersing a magnesium alloy implant in an alkaline solution to form an oxide-coated implant; and
coating the oxide-coated implant with a PCL-lawsone solution to form a coated orthopedic implant.

13. The method of claim 12, further comprising applying a polymer protective layer to the coated orthopedic implant.

14. The method of claim 13, wherein the polymer protective layer comprises PCL.

15. The method of claim 12, wherein the alkaline solution is about 0.1-5 M NaOH.

16. The method of claim 12, wherein the PCL-lawsone solution comprises about 1% w/w of lawsone to PCL.
